(19) United States
(12) Patent Application Publication
(10) Pub. No.: US 2026/0199258 A1
(43) Pub. Date: Jul. 16, 2026

(54) POLYDOPAMINE ENCAPSULATED MATERIALS AND METHODS OF MAKING AND USING THEREOF

(71) Applicant: OHIO STATE INNOVATION FOUNDATION, Columbus, OH (US)

(72) Inventors: Andre PALMER, Columbus, OH (US); Chintan SAVLA, Columbus, OH (US); Ethan POZY, Columbus, OH (US)

(21) Appl. No.: 19/128,883

(22) PCT Filed: Nov. 10, 2023
(86) PCT No.: PCT/US2023/079410

§ 371 Date: May 9, 2025

Related U.S. Application Data
(60) Provisional application No. 63/424,327, filed on Nov. 10, 2022.

(51) Int. Cl. A61K 9/51; A61K 38/42; B82Y 5/00

(52) U.S. Cl. CPC A61K 9/5192; A61K 9/5146; A61K 38/42; B82Y 5/00

(57) ABSTRACT

Disclosed are methods for encapsulating pH sensitive cargos, including oxygen binding proteins, in a polydopamine shell.

### CROSS REFERENCES TO RELATED APPLICATIONS

This application claims benefit of U.S. Provisional Application No. 63/424,327, filed Nov. 10, 2022, which is hereby incorporated herein by reference in its entirety.

### STATEMENT REGARDING FEDERALLY SPONSORED RESEARCH OR DEVELOPMENT

This invention was made with government support under grant numbers R01HL126945, R01HL138116, and R01EB021926 awarded by the National Institutes of Health. The government has certain rights in the invention.

### BACKGROUND

Limitations within the blood supply system, notably the tightly controlled environmental conditions under which red blood cells (RBCs) must be stored and the limited ex vivo shelf life of donated RBC units, has restricted the use of traditional blood components in critical scenarios where proper storage conditions are not feasible, such as emergency and battlefield scenarios. Researchers have investigated hemoglobin (Hb)-based oxygen carriers (HBOCs) as RBCs substitutes for use in transfusion medicine. One goal of this area of research is to develop an HBOC that may be produced independently of the need for human Hb derived from human donors, and without the current limitations of transfusion medicine including blood type specificity, limited ex vivo shelf-life, and specific storage conditions. HBOCs devoid of lipid components present promising solutions to each of these challenges. In the absence of an RBC membrane, HBOCs do not carry cell surface markers that promote the immune response caused by the transfusion of incompatible blood types and may be frozen to extend ex vivo storage lifetime.

However, despite decades of research, no HBOC has cleared phase III clinical trials and attained commercial availability. The challenges associated with transfusing unmodified cell-free Hb include side-effects related to Hb extravasation into the tissue space, quenching of the vaso-regulator nitric oxide (NO), which results in vasoconstriction and systemic hypertension. Accordingly, there remains a need for new and promising HBOCs.

### SUMMARY

One potential strategy for developing new HBOCs is to increase the molecular diameter of a Hb-based material to prevent extravasation via approaches such as inter-molecular cross-linking and polymerization, surface conjugation, liposome encapsulation, and nanoparticle formulation among others.

An intriguing potential HBOC is Lumbricus terrestris erythrocruorin (LtEc), the acellular mega-hemoglobin derived from annelids. The large 3.6 MDa molecular weight (MW) and ~30 nm diameter of LtEc makes the protein a promising precursor for synthesis of HBOCs. Furthermore, LtEc contains 144 oxygen-binding heme sites compared to 4 for human Hb (hHb). Due to its large size, LtEc does not extravasate into surrounding tissues, eliminating the side-effects elicited by small diameter HBOCs. LtEc also possesses a lower rate of auto-oxidation compared to hHb, and thus retains its functional non-oxidized ferrous state for up to several days in the circulation. LtEc's functional circulation time is instead limited by the potential immune response from having a non-mammalian protein in the circulation. To improve its circulation time, research has been conducted attempting to mask the potential immune response of LtEc by surface coating it with biocompatible polymers. For example, both oxidized dextran (Odex) and polyethylene glycol (PEG) have been used to surface coat LtEc.

One bioinspired surface coating gaining interest in biomedical applications is the mussel-inspired polydopamine (PDA), a polymer synthesized by the self-polymerization of the neurotransmitter dopamine (DA). PDA is a desirable surface coating due to its' hydrophilic and biocompatible properties, and has been investigated for biomedical applications from nano-scale protein coating to macroscale medical device coating. The ability of PDA-coatings to act as a reducing agent has further generated interest in surface coating of Hb-particles. The most widely used method for PDA synthesis and surface coating is the self-polymerization of dopamine (DA) under alkaline conditions (pH >8.0). Unlike smaller tetrameric Hbs, LtEc is not stable under these reaction conditions: the oligomeric structure of LtEc begins to dissociate into its monomer and trimer units, which are similar in size to hHb. Therefore, in order to preserve the structure and size of LtEc during the generation of the PDA coating, pH-independent methods must be investigated.

PDA can be synthesized under physiological conditions using the photocatalyst 9-mesityl-10-methylacridinium tetrafluoroborate (Acr-Mes), which creates a controlled level of oxidants to drive the formation of PDA from DA. Unlike many conventional methods of DA polymerization, which employ the addition of strong oxidants that may drive oxidation of Hb into methemoglobin (metHb) or alkaline conditions that may cause LtEc to dissociate into smaller protein species, this photoredox catalytic method may be conducted at neutral (pH 7.0) or physiological (pH 7.4) conditions without the presence of a strong oxidant. Hence, as described herein, this photoredox catalytic method can be used to coat LtEc with PDA (PDA-LtEc), something not previously possible with other methods of PDA synthesis. Successful synthesis of PDA-LtEc can provide an approach for PDA surface coating that can be extremely useful for HBOCs and other proteins and particles that may be sensitive to the reaction conditions that drive pH-dependent PDA synthesis.

Accordingly, described herein are methods for encapsulating a pH sensitive cargo in a polydopamine shell. These methods can comprise contacting the cargo with dopamine monomers and a photoredox catalyst to form a reaction mixture; and irradiating the reaction mixture with actinic radiation, thereby polymerizing the dopamine monomers to form a shell of polydopamine encapsulating the cargo.

In some aspects, the cargo can comprise a nanoparticle or microparticle.

In some aspects, the cargo can comprise a biomolecule, such as a protein. In certain aspects, the cargo can comprise an oxygen binding protein, such as hemoglobin, myoglobin, erythrocruorin, hemocyanin, hemerythrin, chlorocruorin, vanabin, pinnaglobin, leghemoglobin, a polymerized version thereof and/or a polymer-conjugated version thereof.

In certain aspects, the cargo comprises an erythrocruorin, such as an annelid erythrocruorin.

In some aspects, contacting the cargo with the dopamine monomers and the photoredox catalyst can comprise stirring a solution or dispersion comprising the cargo, the dopamine monomers, and the photoredox catalyst. The solution or dispersion can comprise an aqueous solution or dispersion. In certain aspects, the aqueous solution or dispersion can have a pH of from 6 to 8, such as a pH of from 6.2 to 7.8, a pH of from 6.4 to 7.8, or a pH of from 6.5 to 7.4.

In some aspects, the photoredox catalyst can be chosen from N-(4-(10H-phenothiazin-10-yl)phenyl)acrylamide, perylene, perylene diimide, 10-phenylphenothiazine, 2,3-dichloro-5,6-dicyano-p-benzoquinone, eosin Y, fluorescein, rose Bengal, methylene blue, 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, bis(2,2′-bipyridine)-(5-aminophenanthroline)ruthenium bis(hexafluorophosphate), tris(bipyridine)ruthenium(II) chloride, tris(bipyridine)ruthenium(II) hexafluorophosphate, tris-(bipyrazine)ruthenium(II) hexafluorophosphate, tris-(phenanthroline)ruthenium(II) chloride, tris-(bipyrimidine)ruthenium(II) chloride, bis-(2-(2′,4′-difluorophenyl)-5-trifluoromethylpyridine)(di-tert-butylbipyridine)iridium(III) hexafluorophosphate, bis-(2-phenylpyridine)(di-tert-butylbipyridine)iridium(III) hexafluorophosphate, and bis(2,9-di(para-anisyl)-1,10-phenanthroline)copper(I) chloride, and combinations thereof. In certain aspects, the photoredox catalyst can comprise 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, or a combination thereof.

In some aspects, the actinic radiation can have a wavelength of from 200 nm to 1000 nm.

In some aspects, the reaction mixture can be held at a temperature of from 20° C. to 30° C.

In some aspects, the method can further comprise, following irradiation, filtering the cargo encapsulated in the polydopamine shell by ultrafiltration against a filtration membrane having a pore size that separates low molecular weight contaminants from the cargo encapsulated in the polydopamine shell.

In certain aspects, the ultrafiltration can comprise tangential-flow filtration.

In certain aspects, the filtration membrane can be rated for retaining solutes having a molecular weight greater than 100 kDa, thereby forming a retentate fraction comprising the cargo encapsulated in the polydopamine shell and a permeate fraction comprising low molecular weight contaminants having a molecular weight less than 100 kDa.

Also provided herein are methods for making a polydopamine-encapsulated oxygen carrier. These methods can comprise contacting an oxygen binding protein with dopamine monomers and a photoredox catalyst to form a reaction mixture; and irradiating the reaction mixture with actinic radiation, thereby polymerizing the dopamine monomers to form the polydopamine-encapsulated oxygen carrier.

In some aspects, the oxygen binding protein can be pH sensitive.

In some aspects, the oxygen binding protein can comprise hemoglobin, myoglobin, erythrocruorin, hemocyanin, hemerythrin, chlorocruorin, vanabin, pinnaglobin, leghemoglobin, a polymerized version thereof and/or a polymer-conjugated version thereof. In certain aspects, the oxygen binding protein comprises an erythrocruorin, such as an annelid erythrocruorin.

In some aspects, contacting the oxygen binding protein with the dopamine monomers and the photoredox catalyst can comprise stirring a solution or dispersion comprising the oxygen binding protein, the dopamine monomers, and the photoredox catalyst. The solution or dispersion can comprise an aqueous solution or dispersion. In certain aspects, the aqueous solution or dispersion can have a pH of from 6 to 8, such as a pH of from 6.2 to 7.8, a pH of from 6.4 to 7.8, or a pH of from 6.5 to 7.4.

In some aspects, the photoredox catalyst can be chosen from N-(4-(10H-phenothiazin-10-yl)phenyl)acrylamide, perylene, perylene diimide, 10-phenylphenothiazine, 2,3-dichloro-5,6-dicyano-p-benzoquinone, eosin Y, fluorescein, rose Bengal, methylene blue, 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, bis(2,2′-bipyridine)-(5-aminophenanthroline)ruthenium bis(hexafluorophosphate), tris(bipyridine)ruthenium(II) chloride, tris(bipyridine)ruthenium(II) hexafluorophosphate, tris-(bipyrazine)ruthenium(II) hexafluorophosphate, tris-(phenanthroline)ruthenium(II) chloride, tris-(bipyrimidine)ruthenium(II) chloride, bis-(2-(2′,4′-difluorophenyl)-5-trifluoromethylpyridine)(di-tert-butylbipyridine)iridium(III) hexafluorophosphate, bis-(2-phenylpyridine)(di-tert-butylbipyridine)iridium(III) hexafluorophosphate, and bis(2,9-di(para-anisyl)-1,10-phenanthroline)copper(I) chloride, and combinations thereof. In certain aspects, the photoredox catalyst can comprise 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, or a combination thereof.

In some aspects, the actinic radiation can have a wavelength of from 200 nm to 1000 nm.

In some aspects, the reaction mixture can be held at a temperature of from 20° C. to 30° C.

In some aspects, the method can further comprise, following irradiation, filtering the polydopamine-encapsulated oxygen carrier by ultrafiltration against a filtration membrane having a pore size that separates low molecular weight contaminants from the polydopamine-encapsulated oxygen carrier.

In certain aspects, the ultrafiltration can comprise tangential-flow filtration.

In certain aspects, the filtration membrane can be rated for retaining solutes having a molecular weight greater than 100 kDa, thereby forming a retentate fraction comprising the polydopamine-encapsulated oxygen carrier and a permeate fraction comprising low molecular weight contaminants having a molecular weight less than 100 kDa.

Also provided are polydopamine-encapsulated oxygen carriers made by the methods described herein, as well as compositions (e.g., blood substitutes) comprising these carriers.

Also provided are particles comprising erythrocruorin encapsulated in a polydopamine shell, as well as compositions (e.g., blood substitutes) comprising a population of these particles.

Also provided herein are methods of treating a subject by administering the particles and compositions described herein. These compositions and particles can be administered, for example, to treat a subject who is suffering from a loss of blood due to injury, hemolytic anemia, equine infectious anemia, feline infectious anemia, a bacterial infection, Factor IV fragmentation, hypersplenation, splenomegaly, hemorrhagic syndrome, hypoplastic anemia, aplastic anemia, idiopathic immune hemadytic conditions, iron deficiency, isoimmune hemdytic anemia, microangiopathic hemolytic anemia, parasitism, or any combination thereof.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

FIG. 1. Reaction schematic showing increasing PDA surface coating of LtEc with increasing reaction time in the presence of photocatalyst and light source and subsequent removal of reagents and byproducts with a 50 kDa TFF module.

FIG. 2. Photograph of PDA-LtEc synthesis reaction at t=0 hour, 1 hour, 2 hour, 3 hour, and 5 hour timepoints.

FIG. 3. Representative elution profile of LtEc and PDA-LtEc (2-, 5-, and 16-hour reactions) analyzed using SEC-HPLC. A broadened left shoulder was observed for PDA-LtEc reacted for t=5 hours, and an increase in peak elution time was observed for PDA-LtEc reacted for t=16 hours.

FIG. 4. Representative particle size distributions of LtEc and PDA-LtEc (2, 5, and 16 hours) from DLS measurements.

FIG. 5. Representative oxygen equilibrium curves of LtEc and PDA-LtEc (2, 5, 16 hours). No changes in P₅₀ were found to be statistically significant. The cooperativity coefficient of PDA-LtEc (16 hours) was significantly reduced compared to LtEc (p<0.05).

FIG. 6. Representative oxygen offloading kinetics of LtEc and PDA-LtEc derivatives.

FIG. 7. Absorbance of the FRAP solution demonstrating the reductive capacity of PDA-LtEc with increasing reduction of Fe³⁺ to Fe²⁺ as the PDA reaction time increases from 2 hours to 5 hours to 16 hours. *Indicates statistically significant difference between groups (p<0.05).

FIG. 8. ABTS free-radical scavenging assay demonstrating the oxidation reduction capacity of PDA-LtEc with decreasing absorbance of ABTS⁺ cations. *Indicates statistical significance between groups (p<0.05).

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Methods

Described herein are methods for encapsulating a pH sensitive cargo in a polydopamine shell. Encapsulation, as used herein, generated refers to the inclusion or containment of a cargo within a surrounding polydopamine material (a shell). In certain embodiments, encapsulation can be complete (also referred to herein as full encapsulation or fully encapsulated). In these embodiments, the shell can completely cover the cargo, such that none of the cargo is exposed or uncovered by the polydopamine shell. In other embodiments, encapsulation can be less than complete (also referred to herein as partial encapsulation or partially encapsulated). Methods for encapsulating a pH sensitive cargo in a polydopamine shell can comprise contacting the cargo with dopamine monomers and a photoredox catalyst to form a reaction mixture; and irradiating the reaction mixture with actinic radiation, thereby polymerizing the dopamine monomers to form a shell of polydopamine encapsulating the cargo.

The pH sensitive cargo can include a cargo that decomposes, denatures, or otherwise irreversibly loses its structure and/or biological activity if incubated for two or more hours in an aqueous solution having a pH of less than 6 or a solution having a pH of greater than 8. In some embodiments, the cargo can comprise a nanoparticle or microparticle. In some embodiments, the cargo can comprise a biomolecule, such as a protein. In certain embodiments, the cargo can comprise an oxygen binding protein, such as hemoglobin, myoglobin, erythrocruorin, hemocyanin, hemerythrin, chlorocruorin, vanabin, pinnaglobin, leghemoglobin, a polymerized version thereof and/or a polymer-conjugated version thereof. In certain embodiments, the cargo comprises an erythrocruorin, such as an annelid erythrocruorin. Suitable cargos include those described in more detail below.

In some embodiments, contacting the cargo with the dopamine monomers and the photoredox catalyst can comprise stirring a solution or dispersion comprising the cargo, the dopamine monomers, and the photoredox catalyst. The solution or dispersion can comprise an aqueous solution or dispersion. In certain embodiments, the aqueous solution or dispersion can have a pH of from 6 to 8, such as a pH of from 6.2 to 7.8, a pH of from 6.4 to 7.8, or a pH of from 6.5 to 7.4.

As used herein, the term “photoredox catalyst” or “photocatalyst” refers to a catalyst that, when exposed to light, is able to cause oxidation or reduction of another compound via single-electron transfer events. The photoredox catalyst will also be oxidized or reduced as a result of this process (i.e., when the other compound is oxidized or reduced, the photoredox catalyst will be reduced or oxidized, respectively). In certain embodiments, the photoredox catalyst, when exposed to light, is capable of triggering or initiating radical polymerization of the monomer (e.g., dopamine monomers) by causing the initiator or iniferter to form a radical which can initiate radical polymerization of the monomer. Common photoredox catalysts useful in the methods described herein include, but are not limited to N-(4-(10H-phenothiazin-10-yl)phenyl)acrylamide, perylene, perylene diimide, 10-phenylphenothiazine, 2,3-dichloro-5,6-dicyano-p-benzoquinone, eosin Y, fluorescein, rose Bengal, methylene blue, 9-mesityl-2,7-dimethyl-10-phenylacridinium tetrafluoroborate, 9-mesityl-10-methylacridinium perchlorate, 9-mesityl-10-methylacridinium tetrafluoroborate, 9-mesityl-10-phenylacridinium tetrafluoroborate, bis(2,2′-bipyridine)-(5-aminophenanthroline)ruthenium bis(hexafluorophosphate), tris(bipyridine)ruthenium(II) chloride, tris(bipyridine)ruthenium(II) hexafluorophosphate, tris-(bipyrazine)ruthenium(II) hexafluorophosphate, tris-(phenanthroline)ruthenium(II) chloride, tris-(bipyrimidine)ruthenium(II) chloride, bis-(2-(2′,4′-difluorophenyl)-5-trifluoromethylpyridine)(di-tert-butylbipyridine)iridium(III) hexafluorophosphate, bis-(2-phenylpyridine)(di-tert-butylbipyridine)iridium(III) hexafluorophosphate, and bis(2,9-di(para-anisyl)-1,10-phenanthroline)copper(I) chloride.

In some embodiments, the photoredox catalyst can be chosen from N-(4-(10H-phenothiazin-10-yl)phenyl)acrylamide, perylene, perylene diimide, 10-phenylphenothiazine, 2,3-dichloro-5,6-dicyano-p-benzoquinone, eosin Y, fluorescein, rose Bengal, methylene blue, 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, bis(2,2′-bipyridine)-(5-aminophenanthroline)ruthenium bis(hexafluorophosphate), tris(bipyridine)ruthenium(II) chloride, tris(bipyridine)ruthenium(II) hexafluorophosphate, tris-(bipyrazine)ruthenium(II) hexafluorophosphate, tris-(phenanthroline)ruthenium(II) chloride, tris-(bipyrimidine)ruthenium(II) chloride, bis-(2-(2′,4′-difluorophenyl)-5-trifluoromethylpyridine)(di-tert-butylbipyridine)iridium(III) hexafluorophosphate, bis-(2-phenylpyridine)(di-tert-butylbipyridine)iridium(III) hexafluorophosphate, and bis(2,9-di(para-anisyl)-1,10-phenanthroline)copper(I) chloride, and combinations thereof. In certain embodiments, the photoredox catalyst can comprise 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, or a combination thereof.

In some embodiments, the actinic radiation can have a wavelength of from 200 nm to 1000 nm.

In some embodiments, the reaction mixture can be held at a temperature of from 20° C. to 30° C.

In some embodiments, the method can further comprise, following irradiation, filtering the cargo encapsulated in the polydopamine shell by ultrafiltration against a filtration membrane having a pore size that separates low molecular weight contaminants from the cargo encapsulated in the polydopamine shell. As used herein, the term “ultrafiltration” is used for processes employing membranes rated for retaining solutes having a molecular weight between about 1 kDa and 1000 kDa.

In certain embodiments, the ultrafiltration can comprise tangential-flow filtration. As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is processed (e.g., continually processed) downstream.

In certain embodiments, the filtration membrane can be rated for retaining solutes having a molecular weight greater than 100 kDa, thereby forming a retentate fraction comprising the cargo encapsulated in the polydopamine shell and a permeate fraction comprising low molecular weight contaminants having a molecular weight less than 100 kDa.

Also provided herein are methods for making a polydopamine-encapsulated oxygen carrier. These methods can comprise contacting an oxygen binding protein with dopamine monomers and a photoredox catalyst to form a reaction mixture; and irradiating the reaction mixture with actinic radiation, thereby polymerizing the dopamine monomers to form the polydopamine-encapsulated oxygen carrier.

In some embodiments, the oxygen binding protein can be pH sensitive.

In some embodiments, the oxygen binding protein can comprise hemoglobin, myoglobin, erythrocruorin, hemocyanin, hemerythrin, chlorocruorin, vanabin, pinnaglobin, leghemoglobin, a polymerized version thereof and/or a polymer-conjugated version thereof. In embodiments aspects, the oxygen binding protein comprises an erythrocruorin, such as an annelid erythrocruorin.

In some embodiments, contacting the oxygen binding protein with the dopamine monomers and the photoredox catalyst can comprise stirring a solution or dispersion comprising the oxygen binding protein, the dopamine monomers, and the photoredox catalyst. The solution or dispersion can comprise an aqueous solution or dispersion. In certain embodiments, the aqueous solution or dispersion can have a pH of from 6 to 8, such as a pH of from 6.2 to 7.8, a pH of from 6.4 to 7.8, or a pH of from 6.5 to 7.4.

In some embodiments, the photoredox catalyst can be chosen from N-(4-(10H-phenothiazin-10-yl)phenyl)acrylamide, perylene, perylene diimide, 10-phenylphenothiazine, 2,3-dichloro-5,6-dicyano-p-benzoquinone, eosin Y, fluorescein, rose Bengal, methylene blue, 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, bis(2,2′-bipyridine)-(5-aminophenanthroline)ruthenium bis(hexafluorophosphate), tris(bipyridine)ruthenium(II) chloride, tris(bipyridine)ruthenium(II) hexafluorophosphate, tris-(bipyrazine)ruthenium(II) hexafluorophosphate, tris-(phenanthroline)ruthenium(II) chloride, tris-(bipyrimidine)ruthenium(II) chloride, bis-(2-(2′,4′-difluorophenyl)-5-trifluoromethylpyridine)(di-tert-butylbipyridine)iridium(III) hexafluorophosphate, bis-(2-phenylpyridine)(di-tert-butylbipyridine)iridium(III) hexafluorophosphate, and bis(2,9-di(para-anisyl)-1,10-phenanthroline)copper(I) chloride, and combinations thereof. In certain embodiments, the photoredox catalyst can comprise 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, or a combination thereof.

In some embodiments, the actinic radiation can have a wavelength of from 200 nm to 1000 nm.

In some embodiments, the reaction mixture can be held at a temperature of from 20° C. to 30° C.

In some embodiments, the method can further comprise, following irradiation, filtering the polydopamine-encapsulated oxygen carrier by ultrafiltration against a filtration membrane having a pore size that separates low molecular weight contaminants from the polydopamine-encapsulated oxygen carrier.

In certain embodiments, the ultrafiltration can comprise tangential-flow filtration.

In certain embodiments, the filtration membrane can be rated for retaining solutes having a molecular weight greater than 100 kDa, thereby forming a retentate fraction comprising the polydopamine-encapsulated oxygen carrier and a permeate fraction comprising low molecular weight contaminants having a molecular weight less than 100 kDa.

Example Cargos and/or Oxygen Carriers

As discussed above, the methods described herein can be used to encapsulate a wide range of cargos, including oxygen carriers, in polydopamine. While example cargos are described below, one of ordinary skill in the art will understand that the methods described herein can be applied to encapsulate a range of cargos, including small molecules, nanoparticles, microparticles, and biomolecules (e.g., proteins and nucleic acids).

In some embodiments, the cargo can comprise a pH sensitive cargo. pH sensitive cargo can include a cargo that decomposes, denatures, or otherwise irreversibly loses its structure and/or biological activity if incubated for two or more hours in an aqueous solution having a pH of less than 6 or a solution having a pH of greater than 8.

In certain embodiments, the cargo can comprise an oxygen binding protein, such as hemoglobin, myoglobin, erythrocruorin, hemocyanin, hemerythrin, chlorocruorin, vanabin, pinnaglobin, leghemoglobin, a polymerized version thereof and/or a polymer-conjugated version thereof.

In certain embodiments, the cargo comprises an erythrocruorin, such as an annelid erythrocruorin (e.g., a polychaeta erythrocruorin, an oligochaete erythrocruorin (including earthworm, i.e. Lumbricus terrestris), or a hirudinea erythrocruorin. In some embodiments, the erythrocruorin can be an arthropod erythrocruorin. In some embodiments, the erythrocruorin can be an insect erythrocruorin. In some embodiments, the erythrocruorin can be a purified Lumbricus terrestris erythrocruorin.

In certain embodiments, the cargo comprises a purified Lumbricus terrestris erythrocruorin.

In other examples, the cargo can comprise a synthetic hemoglobin-based oxygen carrier.

In some embodiments, the synthetic hemoglobin-based oxygen carrier comprises a modified hemoglobin. Hemoglobin (Hb) is the oxygen-carrying component of blood that circulates through the bloodstream inside small enucleate cells known as erythrocytes or red blood cells. It is a protein comprised of four associated polypeptide chains that bear prosthetic groups known as hemes. The structure of hemoglobin is well known and described in Bunn & Forget, eds., Hemoglobin: Molecular, Genetic and Clinical Aspects (W. B. Saunders Co., Philadelphia, Pa.: 1986) and Fermi & Perutz “Hemoglobin and Myoglobin,” in Phillips and Richards, Atlas of Molecular Structures in Biology (Clarendon Press: 1981). As blood circulates through the lungs, the oxygen present in the alveolar capillaries diffuses through the alveolar membrane and acts to convert virtually all of the hemoglobin within the red cells to a reversible molecular complex known as oxyhemoglobin. During this oxygenation process, the red blood cells become cherry red in color. Because the association of the oxygen and hemoglobin molecules within the red cells is reversible, the oxygen molecules are gradually released from the hemoglobin molecules (or from the red blood cells) when blood reaches the tissue capillaries. Eventually, the oxygen molecules diffuse into the tissues and is consumed by metabolism. As the oxyhemoglobin releases its bound oxygen, the red cells become purple in color.

As used herein, the term “hemoglobin” refers to the iron-containing oxygen-transport metalloprotein in the red blood cells of all vertebrates. Hemoglobin can be obtained from a variety of mammalian sources, such as, for example, human, or bovine (genus bos), or bison (genus bison), or ovine (genus ovis), or porcine (genus sus) sources, or other vertebrates or as transgenically-produced hemoglobin. Alternatively, the hemoglobin for use in the methods and compositions described herein can be synthetically produced by a bacterial cell, or more preferably, by a yeast cell, mammalian cell, or insect cell expression system (Hoffman, S. J. et al., U.S. Pat. No. 5,028,588 and Hoffman, et al., WO 90/13645, both herein incorporated by reference). Alternatively, hemoglobin can be obtained from transgenic animals; such animals can be engineered to express non-endogenous hemoglobin (Logan, J. S. et al. PCT Application No. PCT/US92/05000; Townes, T. M. et al., PCT Application No. PCT/US/09624, both herein incorporated by reference in their entirety).

Hemoglobin can also encompass genetically modified and/or recombinantly produced hemoglobin as well as chemically treated or surface decorated hemoglobins either in their dimeric, or tetrameric or variously polymerized forms. Expression of various recombinant hemoglobins has been achieved. Such expression methods include individual globin expression as described, for example, in U.S. Pat. No. 5,028,588, and di-alpha globin expression created by joining two alpha globins with a glycine linker through genetic fusion coupled with expression of a single beta globin gene to produce a pseudotetrameric hemoglobin molecule as described in WO 90/13645 and Looker et al., Nature 356:258 260 (1992). Other modified recombinant hemoglobins are disclosed in PCT Publication WO 96/40920. Similar to other heterologous proteins expressed in E. coli, recombinant hemoglobins have N-terminal methionines, which in some recombinant hemoglobins replace the native N-terminal valines.

In some embodiments, the hemoglobin is from a mammalian, invertebrate, or recombinant source. In certain embodiments, the hemoglobin is from a mammalian source. For example, the hemoglobin can comprise bovine hemoglobin, procine hemoglobin, or human hemoglobin. In certain embodiments, the hemoglobin can comprise recombinantly produced hemoglobin. In other embodiments, the hemoglobin can comprise chemically or genetically modified hemoglobin that, for example, prevent dissociation of the hemoglobin molecule or modify the oxygen-binding affinity.

In some embodiments, the synthetic hemoglobin-based oxygen carrier comprises polymerized hemoglobin.

In some embodiments, the synthetic hemoglobin-based oxygen carrier comprises a polymer-conjugated hemoglobin.

In some embodiments, the synthetic hemoglobin-based oxygen carrier comprises a population of hemoglobin nanoparticles.

In some embodiments, the synthetic hemoglobin-based oxygen carrier comprises an encapsulated hemoglobin (e.g., hemoglobin encapsulated in a carrier particle).

In some aspects, the synthetic hemoglobin-based oxygen carrier is present in an amount of from 25 mg/mL to 200 mg/mL, such as from 40 mg/mL to 100 mg/mL.

In some embodiments, the synthetic hemoglobin-based oxygen carrier can comprise polymerized hemoglobin. The term “polymerized,” as used herein with respect to hemoglobin, encompasses both inter-molecular and intramolecular polyhemoglobin, with at least 50%, preferably greater than about 95%, of the polymerized hemoglobin of greater than tetrameric form. The polymerized hemoglobin can be prepared by polymerizing or cross-linking hemoglobin with a multifunctional cross-linking agent. Preferably, the polymerized hemoglobin is substantially soluble in aqueous fluids having a pH of 6 to 9 and in physiological fluids. Suitable examples of cross-linking agents are disclosed in U.S. Pat. No. 4,001,200, the entire teachings of which are incorporated herein by reference.

Suitable specific examples of the cross-linking agents include compounds having an aldehyde or dialdehyde functionality, such as glutaraldehyde, formaldehyde, paraformaldehyde, formaldehyde activated ureas such as 1,3-bis(hydroxymethyl)urea, N,N′-di(hydroxymethyl) imidazolidinone prepared from formaldehyde condensation with a urea; compounds bearing a functional isocyanate or isothiocyanate group, such as diphenyl-4,4′-diisothiocyanate-2,2′-disulfonic acid, toluene diisocyanate, toluene-2-isocyanate-4-isothiocyanate, 3-methoxydiphenylmethane-4,4′-diisocyanate, propylene diisocyanate, butylene diisocyanate, and hexamethylene diisocyanate; esters and thioesters activated by strained thiolactones; hydroxysuccinimide esters; halogenated carboxylic acid esters; and imidates. Other examples of the cross-linking agents include derivatives of carboxylic acids and carboxylic acid residues of hemoglobin activated in situ to give a reactive derivative of hemoglobin that will cross-link with the amines of another hemoglobin. Examples of the carboxylic acids include citric, malonic, adipic and succinic acids. Carboxylic acid activators include thionyl chloride, carbodiimides, N-ethyl-5-phenyl-isoxazolium-3′-sulphonate (Woodward's reagent K), N,N′-carbonyldiimidazole, N-t-butyl-5-methylisoxazolium perchlorate (Woodward's reagent L), 1-ethyl-3-dimethyl aminopropylcarbodiimde, and 1-cyclohexyl-3-(2-mopholinoethyl) carbodiimide metho-p-toluene sulfonate. The cross-linking reagent can be a dialdehyde precursor that readily forms a bifunctional dialdehyde in the reaction medium. Suitable dialdehyde precursors include acrolein dimer or 3,4-dihydro-1,2-pyran-2-carboxaldehyde which undergoes ring cleavage in an aqueous environment to give alpha-hydroxy-adipaldehyde. Other precursors, which on hydrolysis yield a cross-linking reagent, include 2-ethoxy-3,4-dihydro-1,2-pyran which gives glutaraldehyde, 2-ethoxy-4-methyl-3,4-dihydro-1,2-pyran which yields 3-methyl glutaraldehyde, 2,5-diethoxy tetrahydrofuran which yields succinic dialdehyde and 1,1,3,3-tetraethoxypropane which yields malonic dialdehyde and formaldehyde from trioxane. Exemplary commercially available cross-linking reagents include divinyl sulfone, epichlorohydrin, butadiene diepoxide, ethylene glycol diglycidyl ether, glycerol diglycidyl ether, dimethyl suberimidate dihydrochloride, dimethyl malonimidate dihydrochloride, and dimethyl adipimidate dihydrochloride.

Specific examples of the cross-linking agents include glutaraldehyde, succindialdehyde, activated forms of polyoxyethylene and dextran, ci-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class.

In some embodiments, the polymerized hemoglobin can comprise hemoglobin polymerized by a dialdehyde. As used herein, the “hemoglobin polymerized by a dialdehyde” includes both hemoglobin polymerized by a dialdehyde and hemoglobin polymerized by a dialdehyde precursor that readily forms a bifunctional dialdehyde in the reaction medium. Suitable dialdehyde and dialdehyde precursors are as described above. In some embodiments, the polymerized hemoglobin can comprise hemoglobin polymerized by glutaraldehyde.

The polymerized hemoglobin can be in the tense or relaxed quaternary state, or in between these two quaternary states. In some embodiments, the hemoglobin can be polymerized in the T-state (tense quaternary state). In other embodiments, the hemoglobin can be polymerized in the R-state (relaxed quaternary state).

In some embodiments, the polymerized hemoglobin can be polymer-functionalized. Polymer-functionalized polymerized hemoglobin can comprise a polymer or oligomer covalently conjugated to the polymerized hemoglobin. Any suitable polymer or oligomer can be used. For example, in some embodiments, the polymer or oligomer can comprise a polyalkylene oxide, such as a polyethylene glycol (PEG), a zwitterionic polymer, such as a polycarboxybetaine (PCB) or a polysulfobetaine (PSB), a carbohydrate such as a dextran, or any combination thereof.

In certain embodiments, the hemoglobin can be polyalkylene oxide (PAO)-functionalized. Polyalkylene oxide (PAO)-functionalized hemoglobin comprises hemoglobin or polymerized hemoglobin that has been surface-modified with one or more polyalkylene oxide (PAO) polymers. In this context, “surface-modification” can refer to the covalent attachment of chemical groups (and ultimately PAO polymer chains) to one or more exposed amino acid side chains on the hemoglobin molecule. Modification can increase the molecular size of the hemoglobin.

Examples of suitable polyalkylene oxides include, but are not limited to, polyethylene oxide ((CH₂CH₂O)ₙ), polypropylene oxide ((CH(CH₃)CH₂O)ₙ), polybutylene oxide ((CH(CH₂CH₃)CH₂O)ₙ), and copolymers thereof such as polyethylene/polypropylene oxide copolymers ((CH₂CH₂O)ₙ (CH(CH₃)CH₂O)ₙ). Such copolymers can include random copolymers, alternating copolymers, and block copolymers. The number of PEGs to be added to the polymerized hemoglobin may vary, depending on the size of the PEG.

In certain embodiments, the PAO is polyethylene glycol (PEG). PEGs are polymers of the general chemical formula H(OCH₂CH₂)ₙOH, where n is generally greater than or equal to 4. PEG formulations are usually followed by a number that corresponds to their average molecular weight. For example, PEG-200 has an average molecular weight of 200 and may have a molecular weight range of 190-210. PEGs are commercially available in a number of different forms, and in many instances come preactivated and ready to conjugate to proteins.

In some embodiments, polymerization and/or surface modification can take place when the hemoglobin is in the oxygenated or “R” state. This can be accomplished by allowing the hemoglobin to equilibrate with the atmosphere (or, alternatively, active oxygenation can be carried out) prior to polymerization and/or conjugation. By performing the polymerization and/or conjugation to oxygenated hemoglobin, the oxygen affinity of the resultant hemoglobin can be enhanced.

In one embodiment, the polymerized hemoglobin is polymerized hemoglobin to which malemidyl-activated PEG (“Mal-PEG”) has been conjugated. Such materials may be further referred to by the following formula:

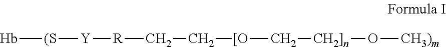

where Hb refers to polymerized hemoglobin, S is a surface thiol group, Y is the succinimido covalent link between Hb and Mal-PEG, R is an alkyl, amide, carbamate or phenyl group (depending on the source of raw material and the method of chemical synthesis), [O—CH₂—CH₂]ₙ are the oxyethylene units making up the backbone of the PEG polymer, where n defines the length of the polymer (e.g., MW=5000), and O—CH₃ is the terminal methoxy group.

In some embodiments, the polymer-functionalized polymerized hemoglobin can have a weight average molecular weight of at least 500 kDa (e.g., at least 600 kDa, at least 700 kDa, at least 750 kDa, at least 800 kDa, at least 900 kDa, at least 1000 kDa, at least 1250 kDa, at least 1500 kDa, or at least 1750 kDa), as determined by size exclusion (SEC) HPLC. In some embodiments, the polymer-functionalized polymerized hemoglobin can have a weight average molecular weight of 2000 kDa or less (e.g., 1750 kDa or less, 1500 kDa or less, 1250 kDa or less, 1000 kDa or less, 900 kDa or less, 800 kDa or less, 750 kDa or less, 700 kDa or less, or 600 kDa or less), as determined by size exclusion (SEC) HPLC.

The polymer-functionalized polymerized hemoglobin can have a weight average molecular weight ranging from any of the minimum values described above to any of the maximum values described above. For example, in some embodiments, the polymer-functionalized polymerized hemoglobin can have a weight average molecular weight of from 500 kDa to 2000 kDa (e.g., from 700 kDa to 1500 kDa).

The polymer-functionalized polymerized hemoglobin can be substantially free of (e.g., can contain less than 5% by weight, less than 1% by weight, or less than 0.5% by weight) low-molecular weight hemoglobin species having a molecular weight of less than 100 kDa, or substantially free of low-molecular weight hemoglobin species having a molecular weight of less than 250 kDa, or substantially free of low-molecular weight hemoglobin species having a molecular weight of less than 500 kDa.

In some embodiments, the polymer-functionalized polymerized hemoglobin exhibits an average hydrodynamic diameter of from 13 nm to 100 nm (e.g., from 13 nm to 50 nm, or from 13 nm to 30 nm), as measured by dynamic light scattering.

In some embodiments, the polymer-functionalized polymerized hemoglobin exhibits a zeta potential of from −40 mV to less than 0 mV (e.g., from −20 mV to less than 0 mV).

In some embodiments, the polymer-functionalized polymerized hemoglobin was polymerized in the T-state (tense quaternary state). In certain embodiments, the polymer-functionalized polymerized hemoglobin exhibits a P₅₀ of from 20 mm Hg to 60 mm Hg, a k_{off,O2} of from 10 s⁻¹ to 40 s⁻¹, or a combination thereof.

In other embodiments, the polymer-functionalized polymerized hemoglobin was polymerized in the R-state (relaxed quaternary state). In certain embodiments, the polymer-functionalized polymerized hemoglobin exhibits a P₅₀ of 1.0±0.5 mm Hg, a k_{off,O2} of from 7 s⁻¹ to 20 s⁻¹, or a combination thereof.

In some embodiments, the polymer-functionalized polymerized hemoglobin can comprise a mixture comprising polymer-functionalized polymerized hemoglobin polymerized in the T-state (tense quaternary state) and polymer-functionalized polymerized hemoglobin polymerized in the R-state (relaxed quaternary state). By varying the relative amount of the polymer-functionalized polymerized hemoglobin polymerized in the T-state (tense quaternary state) and polymer-functionalized polymerized hemoglobin polymerized in the R-state (relaxed quaternary state) present in the compositions, the oxygen transport characteristics of the composition (e.g., P₅₀, k_{off,O2}, etc.) can be tuned.

Methods of making polymerized hemoglobin can comprise: (i) contacting hemoglobin with a multifunctional cross-linking agent to form a solution comprising polymerized hemoglobin; and (ii) filtering the solution comprising polymerized hemoglobin by ultrafiltration (e.g., tangential flow filtration) against a first filtration membrane having a pore size that separates the polymerized hemoglobin from low-molecular weight hemoglobin species, reactants, and reaction byproducts, thereby forming a retentate fraction comprising the polymerized hemoglobin and a permeate fraction comprising impurities. In the case of polymer-functionalized polymerized hemoglobin, these methods can further comprise (iii) covalently conjugating one or more polymers to the polymerized hemoglobin to form a solution comprising a polymer-functionalized polymerized hemoglobin; and (iv) filtering the solution comprising the polymer-functionalized polymerized hemoglobin by ultrafiltration (e.g., tangential flow filtration) against a second filtration membrane having a pore size that separates the polymer-functionalized polymerized hemoglobin from low-molecular weight impurities, reactants, and reaction byproducts, thereby forming a retentate fraction comprising the polymer-functionalized polymerized hemoglobin and a permeate fraction comprising impurities.

In some embodiments, step (i) can comprise deoxygenating the hemoglobin such that substantially all of the hemoglobin is in the T-state (tense quaternary state) prior to contacting the hemoglobin with the multifunctional cross-linking agent. In other embodiments, step (i) can comprise oxygenating the hemoglobin such that substantially all of the hemoglobin is in the R-state (relaxed quaternary state) prior to contacting the hemoglobin with the multifunctional cross-linking agent.

In some embodiments, the multifunctional cross-linking agent can comprise a dialdehyde, such as glutaraldehyde. The multifunctional cross-linking agent and the hemoglobin are present at a molar ratio of dialdehyde:hemoglobin of from 20:1 to 35:1.

In some examples, the hemoglobin utilized in step (i) can further comprise one or more antioxidant proteins which also react with the multifunctional cross-linking agent, thereby becoming co-polymerized with the hemoglobin. The one or more antioxidant proteins can comprise antioxidant proteins present in red blood cells, such as a peroxiredoxin (e.g., peroxiredoxin-1, -2, and/or -6), a superoxide dismutase, a catalase, or a combination thereof. In some examples, step (i) can be performed using a clarified red blood cell lysate which includes a mixture of hemoglobin, antioxidant proteins, and optionally one or more additional proteins found in red blood cells.

The first filtration membrane can be rated for removing solutes having a molecular weight less than the molecular weight of the polymerized hemoglobin. In some examples, the first filtration membrane is rated for removing solutes having a molecular weight of from 1 to 500 kDa, from 1 to 250 kDa, from 1 to 100 kDa, from 1 to 50 kDa, or from 1 to 10 kDa.

In some embodiments, step (ii) can further comprise filtering the solution comprising polymerized hemoglobin by ultrafiltration (e.g., tangential flow filtration) against a third filtration membrane having a pore size that separates the polymerized hemoglobin from high-molecular weight hemoglobin species, reactants, and reaction byproducts, thereby forming a permeate fraction comprising the polymerized hemoglobin and a retentate fraction comprising impurities.

In some examples, the third filtration membrane can be rated for retaining solutes having a molecular weight greater than the molecular weight of the polymerized hemoglobin. For example, the third filtration membrane can be rated for retaining solutes having a molecular weight of at least 500 kDa, at least 750 kDa, at least 1000 kDa, or more. In certain embodiments, the third filtration membrane can have a pore size of at least about 0.1 μm, such as a pore size of about 0.2 μm.

In some embodiments, following step (ii), substantially all of the polymerized hemoglobin has a molecular weight of at least 100 kDa, such as at least 250 kDa or at least 500 kDa. For example, in certain embodiments, following step (ii), substantially all of the polymerized hemoglobin has a molecular weight of from 100 kDa to 10,000 kDa, such as from 100 kDa to 500 kDa, from 100 kDa to 750 kDa, from 100 kDa to 1,000 kDa, from 100 kDa to 5,000 kDa, from 250 kDa to 500 kDa, from 250 kDa to 750 kDa, from 250 kDa to 1,000 kDa, from 250 kDa to 5,000 kDa, 250 kDa to 10,000 kDa, from 500 kDa to 750 kDa, from 500 kDa to 1,000 kDa, from 500 kDa to 5,000 kDa, 500 kDa to 10,000 kDa, from 750 kDa to 1,000 kDa, or from 750 kDa to 5,000 kDa, or from 750 kDa to 10,000 kDa.

Step (iii) can comprise covalently conjugating one or more polyalkylene oxides polymers, such as one or more polyethylene glycol (PEG) polymers, to the polymerized hemoglobin to form a solution comprising a polyalkylene oxide (PAO)-functionalized polymerized hemoglobin, such as a polyethylene glycol (PEG)-functionalized polymerized hemoglobin. For example, in some embodiments, step (iii) can comprise contacting the polymerized hemoglobin with a thiolating reagent (e.g., 2-Iminothiolane, Traut's reagent) and a malemidyl-activated PAO, such as a malemidyl-activated polyethylene glycol (Mal-PEG).

The second filtration membrane can be rated for removing solutes having a molecular weight less than the molecular weight of the PAO-functionalized polymerized hemoglobin. In some examples, the second filtration membrane is rated for removing solutes having a molecular weight of from 1 to 750 kDa, from 1 to 500 kDa, from 1 to 250 kDa, or from 1 to 100 kDa.

In connection with the methods described above, ultrafiltration can comprise direct-flow filtration (DFF), cross-flow or tangential-flow filtration (TFF), or a combination thereof. In certain embodiments, the ultrafiltration can comprise tangential-flow filtration (TFF).

The membranes useful in the filtration steps described herein can be in the form of flat sheets, rolled-up sheets, cylinders, concentric cylinders, ducts of various cross-section and other configurations, assembled singly or in groups, and connected in series or in parallel within the filtration unit. The apparatus can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler F R. Tangential flow filtration for processing cells, proteins, and other biological components. ASM News 1984; 50:299-304. They can be synthetic membranes of either the microporous type or the ultrafiltration type. A microporous membrane has pore sizes typically from 0.1 to 10 micrometers, and can be made so that it retains all particles larger than the rated size. Ultrafiltration membranes have smaller pores and are characterized by the size of the protein that will be retained. They are available in increments from 1000 to 1,000,000 Dalton nominal molecular weight limits.

Generally, the filtration membrane can comprise an ultrafiltration membrane. Ultrafiltration membranes are normally asymmetrical with a thin film or skin on the upstream surface that is responsible for their separating power. They are commonly made of regenerated cellulose, polysulfone or polyethersulfone. In some cases, the filtration membrane can be rated for retaining solutes having a molecular weight of from 1 to 750 kDa, such as from 1 to 500 kDa, from 1 to 250 kDa, from 1 to 100 kDa, or from 1 to 50 kDa.

In some cases, each filtration step can involve filtration through a single filtration membrane. In other cases, because membrane filters are not perfect and may have holes that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane.

Membrane filters for tangential-flow filtration are available as units of different configurations depending on the volumes of liquid to be handled, and in a variety of pore sizes. Particularly suitable for use in the methods described herein, on a relatively large scale, are those known, commercially available tangential-flow filtration units.

The filtration unit useful herein is suitably any unit now known or discovered in the future that serves as an appropriate filtration module, particularly for microfiltration and ultrafiltration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon ultrafiltration system available from Millipore, Bedford, Mass.

In some embodiments, the hemoglobin can be purified using ultrafiltration (e.g., tangential flow filtration) prior to polymerization. For example, in some cases, the hemoglobin can be purified using a multistage tangential flow filtration process, such as that described in Palmer, A. F.; Sun, G.; Harris, D. R. Tangential Flow Filtration of Hemoglobin. Biotechnol. Prog. 2009, 25 (1), 189-199.

In some embodiments, the synthetic hemoglobin-based oxygen carrier can comprise polymer-conjugated hemoglobin. Polymer-conjugated hemoglobin can comprise a polymer or oligomer covalently conjugated to hemoglobin. Any suitable polymer or oligomer can be used. For example, in some embodiments, the polymer or oligomer can comprise a polyalkylene oxide, such as a polyethylene glycol (PEG), a zwitterionic polymer, such as a polycarboxybetaine (PCB) or a polysulfobetaine (PSB), a carbohydrate such as a dextran, or any combination thereof.

In certain embodiments, the polymer-conjugated hemoglobin can be polyalkylene oxide (PAO)-functionalized. Polyalkylene oxide (PAO)-functionalized hemoglobin comprises hemoglobin that has been surface-modified with one or more polyalkylene oxide (PAO) polymers. In this context, “surface-modification” can refer to the covalent attachment of chemical groups (and ultimately PAO polymer chains) to one or more exposed amino acid side chains on the hemoglobin molecule. Modification can increase the molecular size of the hemoglobin.

Examples of suitable polyalkylene oxides include, but are not limited to, polyethylene oxide ((CH₂CH₂O)ₙ), polypropylene oxide ((CH(CH₃)CH₂O)ₙ), polybutylene oxide ((CH(CH₂CH₃)CH₂O)ₙ), and copolymers thereof such as polyethylene/polypropylene oxide copolymers ((CH₂CH₂O)ₙ (CH(CH₃)CH₂O)ₙ). Such copolymers can include random copolymers, alternating copolymers, and block copolymers. The number of PEGs to be added to the polymerized hemoglobin may vary, depending on the size of the PEG.

In certain embodiments, the PAO is polyethylene glycol (PEG). PEGs are polymers of the general chemical formula H(OCH₂CH₂)ₙOH, where n is generally greater than or equal to 4. PEG formulations are usually followed by a number that corresponds to their average molecular weight. For example, PEG-200 has an average molecular weight of 200 and may have a molecular weight range of 190-210. PEGs are commercially available in a number of different forms, and in many instances come preactivated and ready to conjugate to proteins.

In some embodiments, polymerization and/or surface modification can take place when the hemoglobin is in the oxygenated or “R” state. This can be accomplished by allowing the hemoglobin to equilibrate with the atmosphere (or, alternatively, active oxygenation can be carried out) prior to polymerization and/or conjugation. By performing the polymerization and/or conjugation to oxygenated hemoglobin, the oxygen affinity of the resultant hemoglobin can be enhanced.

In some embodiments, polymerization and/or surface modification can take place when the hemoglobin is in the deoxygenated or “T” state. This can be accomplished by allowing the hemoglobin to equilibrate with an inert gas (or, alternatively, active deoxygenation can be carried out) prior to polymerization and/or conjugation. By performing the polymerization and/or conjugation to deoxygenated hemoglobin, the oxygen affinity of the resultant hemoglobin can be reduced.

In one embodiment, the polymer-conjugated hemoglobin is hemoglobin to which malemidyl-activated PEG (“Mal-PEG”) has been conjugated. Such HBOCs may be further referred to by the following formula:

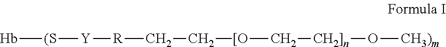

where Hb refers to polymerized hemoglobin, S is a surface thiol group, Y is the succinimido covalent link between Hb and Mal-PEG, R is an alkyl, amide, carbamate or phenyl group (depending on the source of raw material and the method of chemical synthesis), [O—CH₂—CH₂]ₙ are the oxyethylene units making up the backbone of the PEG polymer, where n defines the length of the polymer (e.g., MW=5000), and O—CH₃ is the terminal methoxy group.

In some embodiments, the polymer-conjugated hemoglobin can have a weight average molecular weight of at least 500 kDa (e.g., at least 600 kDa, at least 700 kDa, at least 750 kDa, at least 800 kDa, at least 900 kDa, at least 1000 kDa, at least 1250 kDa, at least 1500 kDa, or at least 1750 kDa), as determined by size exclusion (SEC) HPLC. In some embodiments, the polymer-conjugated hemoglobin can have a weight average molecular weight of 2000 kDa or less (e.g., 1750 kDa or less, 1500 kDa or less, 1250 kDa or less, 1000 kDa or less, 900 kDa or less, 800 kDa or less, 750 kDa or less, 700 kDa or less, or 600 kDa or less), as determined by size exclusion (SEC) HPLC.

The polymer-conjugated hemoglobin can have a weight average molecular weight ranging from any of the minimum values described above to any of the maximum values described above. For example, in some embodiments, the polymer-conjugated hemoglobin can have a weight average molecular weight of from 500 kDa to 2000 kDa (e.g., from 700 kDa to 1500 kDa).

The polymer-conjugated hemoglobin can be substantially free of (e.g., can contain less than 5% by weight, less than 1% by weight, or less than 0.5% by weight) low-molecular weight hemoglobin species having a molecular weight of less than 100 kDa, or substantially free of low-molecular weight hemoglobin species having a molecular weight of less than 250 kDa, or substantially free of low-molecular weight hemoglobin species having a molecular weight of less than 500 kDa.

In some embodiments, the polymer-conjugated hemoglobin exhibits an average hydrodynamic diameter of from 13 nm to 100 nm (e.g., from 13 nm to 50 nm, or from 13 nm to 30 nm), as measured by dynamic light scattering.

In some embodiments, the polymer-conjugated hemoglobin exhibits a zeta potential of from −40 mV to less than 0 mV (e.g., from −20 mV to less than 0 mV).

In some embodiments, the polymer-conjugated hemoglobin was polymerized in the T-state (tense quaternary state). In certain embodiments, the polymer-conjugated hemoglobin exhibits a P₅₀ of from 20 mm Hg to 60 mm Hg, a k_{off,O2} of from 10 s⁻¹ to 40 s⁻¹, or a combination thereof.

In other embodiments, the polymer-conjugated hemoglobin was polymerized in the R-state (relaxed quaternary state). In certain embodiments, the polymer-conjugated hemoglobin exhibits a P₅₀ of 1.0±0.5 mm Hg, a k_{off,O2} of from 7 s⁻¹ to 20 s⁻¹, or a combination thereof.

In some embodiments, the polymer-conjugated hemoglobin can comprise a mixture comprising polymer-conjugated hemoglobin in the T-state (tense quaternary state) and polymer-conjugated hemoglobin in the R-state (relaxed quaternary state). By varying the relative amount of the T-state (tense quaternary state) and R-state (relaxed quaternary state) present in the compositions, the oxygen transport characteristics of the composition (e.g., P₅₀, k_{off,O2}, etc.) can be tuned.

In some embodiments, the synthetic hemoglobin-based oxygen carrier can comprise hemoglobin nanoparticles.

In some embodiments, the hemoglobin nanoparticles can comprise a plurality of particles comprising one or more proteins; wherein the plurality of particles have an average particle size of less than 1000 nm in diameter; wherein each particle within the plurality of particles comprises at least 25 weight percent hemoglobin based on the total weight of all proteins present in the particles; wherein each particle has an outer surface; wherein the hemoglobin and or other protein molecule present on the outer surface have been substantially crosslinked using a crosslinker; and wherein the oxygen transporting formulation is sufficiently free of surfactant.

In some embodiments, the hemoglobin nanoparticles can comprise a plurality of particles, wherein the plurality of particles has an average particle size of less than 1000 nm in diameter; wherein each particle within the plurality of particles comprises at least 25 weight percent hemoglobin and optionally one or more additional proteins; wherein each particle has an outer surface; wherein the hemoglobin and/or protein molecules present on the outer surface have been substantially crosslinked using a chemical crosslinker; and wherein the oxygen transporting formulation is sufficiently free of surfactant.

In some embodiments, each particle comprises at least 25% by weight hemoglobin and/or optionally other proteins the total protein weight of the particle. In some embodiments, each particle comprises at least 30% by weight hemoglobin and/or optionally other proteins the total protein weight of the particle. In some embodiments, each particle comprises at least 35% by weight hemoglobin and/or optionally other proteins the total protein weight of the particle. In some embodiments, each particle comprises at least 40% by weight hemoglobin and/or optionally other proteins the total protein weight of the particle. In some embodiments, each particle comprises at least 50% by weight hemoglobin and/or optionally other proteins the total protein weight of the particle. In some embodiments, each particle comprises at least 60% by weight hemoglobin and/or optionally other proteins the total protein weight of the particle. In some embodiments, each particle comprises at least 70% by weight hemoglobin and/or optionally other proteins the total protein weight of the particle. In some embodiments, each particle comprises at least 75% by weight hemoglobin and/or optionally other proteins the total protein weight of the particle. In some embodiments, each particle comprises at least 80% by weight hemoglobin and/or optionally other proteins the total protein weight of the particle. In some embodiments, each particle comprises at least 90% by weight hemoglobin and/or optionally other proteins the total protein weight of the particle. In some embodiments, each particle comprises at least 95% by weight hemoglobin and/or optionally other proteins based on the total protein weight of the particle.

In some embodiments, each particle comprises 25% hemoglobin and 75% human serum albumin (HSA) for the protein component of the particle. In some embodiments, each particle comprises 50% hemoglobin and 50% HSA for the protein component of the particle. In some embodiments, each particle comprises 75% hemoglobin and 25% HSA for the protein component of the particle. In some embodiments, each particle comprises 95% or more hemoglobin for the protein component of the particle. In some embodiment, each particle comprises 100% hemoglobin for the protein component of the particle.

In some embodiments, the plurality of particles is characterized in having an average particle size, as determined by dynamic light scattering, of less than 1000 nm. In some embodiments, the plurality of particles is characterized in having an average particle size of less than 900 nm. In some embodiments, the plurality of particles is characterized in having an average particle size of less than 800 nm. In some embodiments, the plurality of particles is characterized in having an average particle size of less than 700 nm. In some embodiments, the plurality of particles is characterized in having an average particle size of less than 600 nm. In some embodiments, the plurality of particles is characterized in having an average particle size of less than 500 nm. In some embodiments, the plurality of particles is characterized in having an average particle size of less than 400 nm. In some embodiments, the plurality of particles is characterized in having an average particle size of less than 300 nm. In some embodiments, the plurality of particles is characterized in having an average particle size of less than 200 nm.

In some embodiments, the plurality of particles is characterized in having an average particle size, as determined by dynamic light scattering, from 100 to 1000 nm. In some embodiments, the plurality of particles is characterized in having an average particle size from 100 to 900 nm. In some embodiments, the plurality of particles is characterized in having an average particle size from 100 to 800 nm. In some embodiments, the plurality of particles is characterized in having an average particle size from 100 to 700 nm. In some embodiments, the plurality of particles is characterized in having an average particle size from 100 to 600 nm. In some embodiments, the plurality of particles is characterized in having an average particle size from 100 to 500 nm. In some embodiments, the plurality of particles is characterized in having an average particle size from 100 to 400 nm. In some embodiments, the plurality of particles is characterized in having an average particle size from 100 to 300 nm. In some embodiments, the plurality of particles is characterized in having an average particle size from 100 to 200 nm.

In some embodiments, the plurality of particles is characterized in having a polydispersity index from about 0 to 0.3. In some embodiments, the plurality of particles is characterized in having a polydispersity index from 0 to 0.25. In some embodiments, the plurality of particles is characterized in having a polydispersity index of 0 to 0.2. In some embodiments, the plurality of particles is characterized in having a polydispersity index of 0 to 0.15. In some embodiments, the plurality of particles is characterized in having a polydispersity index of 0 to 0.1. In some embodiments, the plurality of particles is characterized in having a polydispersity index less than 0.3. In some embodiments, the plurality of particles is characterized in having a polydispersity index less than 0.1.

In some embodiments, the plurality of particles is characterized in having a zeta potential of less than −10 mV. In some embodiments, the plurality of particles is characterized in having a zeta potential of less than −15 mV. In some embodiments, the plurality of particles is characterized in having a zeta potential of less than −20 mV. In some embodiments, the plurality of particles is characterized in having a zeta potential of less than −25 mV. In some embodiments, the plurality of particles is characterized in having a zeta potential of less than −30 mV. In some embodiments, the plurality of particles is characterized in having a zeta potential of less than −35 mV. In some embodiments, the plurality of particles is characterized in having a zeta potential of less than −10 mV, less than −12 mV, less than −14 mV, less than −16 mv, less than −18 mV, less than −20 mV, less than −22 mV, less than −24 mV, less than −26 mV, less than −28 mV, less than −30 mV, less than −32 mV, less than −34 mV, less than −36 mV, or less than −38 mV.

In some embodiments, the plurality of particles is characterized in having a negative zeta potential. In some embodiments, the plurality of particles is characterized in having a zeta potential ranging from −40 mV to −10 mV. In some embodiments, the plurality of particles is characterized in having a zeta potential ranging from −40 mV to −20 mV. In some embodiments, the plurality of particles is characterized in having a zeta potential ranging from −40 mV to −30 mV. In some embodiments, the plurality of particles is characterized in having a zeta potential ranging from −35 mV to −15 mV. In some embodiments, the plurality of particles is characterized in having a zeta potential ranging from −35 mV to −20 mV. In some embodiments, the plurality of particles is characterized in having a zeta potential ranging from −35 mV to −25 mV. In some embodiments, the plurality of particles is characterized in having a zeta potential ranging from −35 mV to −30 mV. In some embodiments, the plurality of particles is characterized in having a zeta potential ranging from −30 mV to −10 mV. In some embodiments, the plurality of particles is characterized in having a zeta potential ranging from −30 mV to −15 mV. In some embodiments, the plurality of particles is characterized in having a zeta potential ranging from −30 mV to −20 mV. In some embodiments, the plurality of particles is characterized in having a zeta potential ranging from −30 mV to −25 mV. In some embodiments, the plurality of particles is characterized in having a zeta potential ranging from −25 mV to −10 mV. In some embodiments, the plurality of particles is characterized in having a zeta potential ranging from −25 mV to −15 mV. In some embodiments, the plurality of particles is characterized in having a zeta potential ranging from −25 mV to −20 mV. In some embodiments, the plurality of particles is characterized in having a zeta potential ranging from −20 mV to −10 mV. In some embodiments, the plurality of particles is characterized in having a zeta potential ranging from −20 mV to −15 mV. In some embodiments, the plurality of particles is characterized in having a zeta potential ranging from −15 mV to −10 mV.

To form the hemoglobin nanoparticles, hemoglobin is dissolved in an aqueous buffer to which a desolvating agent is added. The desolvating agent is a liquid in which hemoglobin is poorly soluble but that is miscible with water. Polar solvents such as alcohols are often well suited to be desolvating agents in this process. As the desolvating agent is added, solute-solute interactions dominated over solute solvent forces, driving nucleating of hemoglobin precipitates. With appropriate conditions, nucleation results in the rapid formation of particles.

Suitable desolvating agents include alcohol desolvating agents, such as methanol, ethanol, propanols, butanols, or mixtures thereof, or acetone. In some embodiments, the addition of concentrated polyethylene glycol solutions (>40% in water) can be used to effect desolvation.

Upon formation of particles, a chemical crosslinker is added to stabilize the particles and fix their size and shape. A suitable chemical crosslinker is used to bind several sites across the surface of the particle, halting particle growth and limiting particle-particle interaction. Depending upon the chemical crosslinker used, it may be prudent to chemically deactivate excess reactants with a suitable quenching agent.

Examples of suitable chemical crosslinkers include polyfunctional agents that will crosslink proteins, for example glutaraldehyde, succindialdehyde, activated forms of polyoxyethylene and dextran, a-hydroxy aldehydes, such as glycoaldehyde, N-maleimido-6-aminocaproyl(2′-nitro-4′-sulfonic acidjphenyl ester, m-maleimidobenzoic acid N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl (4-iodoacetyl)aminobenzoate, sulfosuccinimidyl 4-(p-maleimidophenyl)butyrate, sulfosuccinimidyl 4-(p-maleimidophenyl)butyrate, 1-ethyl-3-(3-dimethylaminopropyljcarbodiimide hydrochloride, N,N′-phenylene dimaleimide, and compounds belonging to the bis-imidate class, the acyl diazide class, or the aryl dihalide class, among others.

When the chemical crosslinker used is not an aldehyde, the nanoparticles formed are stable.

When the chemical crosslinker is an aldehyde, the nanoparticles formed are not stable until mixed with a suitable reducing agent to reduce the less stable bonds to form more stable bonds. Examples of suitable reducing agents include sodium borohydride, sodium cyanoborohydride, sodium dithionite, trimethylamine, t-butylamine, morpholine borane, and pyridine borane. In some embodiments, the reducing agent is also used to reduce reactivity of any residual chemical crosslinker present in the solution to prevent crosslinking between particles.

Any time after precipitation, the surfaces of the particles may be modified with the addition of a surface treatment agent. Depending upon solubility and function of the surface treatment agent in the reaction buffer, it may be prudent to perform the surface modification after removal of the desolvating agent.

Representative examples of surface treatment agents include, but are not limited to: other proteins such as human serum albumin (HSA); oligosaccharides; polysaccharides, such as, for example dextran; a polyelectrolyte such as an ionomer, lignosulfonates, sulfonated tetrafluoroethylene polymers, poly(3,4-ethylenedioxythiophene) polystyrene sulfonate, polyacrylamide, polyacrylic acid, polyallylamine hydrochloride, poly(2-acrylamido-2-methyl-1-propanesulfonic acid), polyaniline, poly(acrylamido-N-propyltrimethylammonium chloride), poly[(3-methylacryloylamino)-propyl]trimethylammonium chloride), polyaspartic acid, polypyridinium salts, polystyrene sulfonate, and sodium polyaspartate; red blood cell membrane components such as red blood cell membrane lipids including cholesterol, phosphatidylcholine, sphingomyelin, phosphatidylethanolamine, phosphoinositol, and phosphatidyl serine or red blood cell membrane proteins such as Band 3, Aquaporin 1, Glutl, Kidd antigen protein, RhAg, Na⁺/K⁺-ATPase, Ca²⁺-ATPase, Na⁺ K⁺ 2C1 cotransporter, Na⁺ Cl cotransporter, Na—H exchanger, K—C1 cotransporter, Gardos channel, ICAM-4, BCAM, Protein 4.1R, Glycophorin C and D, XK, RhD/RhCE, Duffy protein, Adducin, Dematin, flotillins, stomatins (band 7), G-proteins, and b-andrenergic receptors; polyethyleneglycols; and zwitterionic polymers.

The desolvating agent may be removed through a variety of means of buffer exchange known to those skilled in protein or particle modification. Small volumes may be washed via ultracentrifugation, but these techniques do not translate well to clinically meaningful scales of production. Larger batches (e.g. greater than 10 mL) may be washed into fresh buffer quite effectively by means of tangential flow filtration (TFF). A hollow-fiber TFF cartridge with polysulfone membrane pore size of about 50 nm is effective for rapid buffer exchange while retaining particles in the system reservoir. Such a TFF system may also be used concentrate materials to a desired concentration (measured in particle/mL or mg Hb/mL).

### Encapsulated Materials and Compositions Containing Thereof

Also provided are polydopamine-encapsulated oxygen carriers made by the methods described herein, as well as compositions (e.g., blood substitutes) comprising these carriers.

Also provided are particles comprising erythrocruorin encapsulated in a polydopamine shell, as well as compositions (e.g., blood substitutes) comprising a population of these particles.

Artificial blood substitutes are a potential alternative to donor blood and provide several advantages over human donor blood. For example, artificial blood substitutes may be: designed to be free of human red blood cell antigens (i.e., can be administered to individuals possessing any blood group type); readily mass-produced with guaranteed sterility (eliminating the possibility of infectious transmittal or the need for infectious blood screening); designed to have longer storage lifetimes and require less stringent storage conditions than donor blood; and produced at lower costs (e.g., by avoiding the screening and storage costs currently associated with human donor blood units).

The various embodiments provide compositions and methods for developing synthetic whole blood substitutes that may include a polydopamine-encapsulated oxygen carriers described herein and/or a population of particles comprising erythrocruorin encapsulated in a polydopamine shell. Optionally, these blood substitutes may further include a plasma substitute/plasma expander.

In order to be an adequate replacement for donor blood, an ideal artificial blood substitute should replicate blood's ability to transport oxygen to tissues. For example, an ideal artificial blood substitute should be an oxygen therapeutic. An ideal synthetic oxygen therapeutic (i.e., oxygen-carrying artificial blood substitutes) should have normal physiological oxygen-binding properties, be uniform and small size so as to both afford long circulation lifetimes and safe clearance from body, have human bloodlike viscosity and oncotic pressure characteristics so as to preserve shear forces in the microcirculation and enable plasma expansion in the resuscitation of patients, have tunable oxygen release parameters for tissues experiencing normal or low oxygenation, and be free of infectious disease risks associated with intravenous administration.

Various embodiments provide an oxygen-carrying artificial blood substitute that has normal physiological oxygen-binding properties, is uniform and small size, has human bloodlike viscosity and oncotic pressure characteristics, has tunable oxygen release parameters, and is resistant to infectious diseases. The polydopamine-encapsulated oxygen carriers described herein and/or a population of particles comprising erythrocruorin encapsulated in a polydopamine shell described herein can provide for safe and effective oxygen delivery.

While existing hemoglobin-based oxygen therapeutics have numerous advantageous over the perfluorocarbons-based oxygen therapeutics, initial studies involving the infusion of cell-free hemoglobin into animals, showed that free hemoglobin results in significant vasoconstriction and kidney damage. Consequently, hemoglobin-based oxygen carriers (HBOCs) often require stabilizing the hemoglobin molecule in order to eliminate adverse physiological effects while maintaining the physiological oxygen-transporting ability of native cell-free hemoglobin. Existing HBOCs can induce vasoconstriction when transfused into animals due to nitric oxide (NO) sequestration and/or an over-oxygenation auto-regulatory response. Moreover, existing HBOCs generally demonstrate limited circulatory half-lives (usually less than 12 hours) and are only suitable for short-term applications. The various embodiments described herein provide oxygen carriers that can maintain their physiological oxygen-transporting abilities while avoiding the adverse physiological effects associated with hemoglobin and existing HBOCs.

Optionally the blood substitute can further comprise a clotting agent, a drag reducing polymer (e.g., polyethylene glycol (PEG), an anti-inflammatory agent (e.g., a steroid, such as dexamethasone), or a combination thereof.

Example components of these synthetic whole blood substitutes are discussed in more detail below.

### Clotting Agents

The blood substitutes described herein can include a clotting agent. The clotting agent can be any component that can regulate the formation or breakdown of blood clots in vivo.

In some embodiments, the clotting agent can comprise platelets. In some embodiments, the clotting agent can comprise a fibrinolysis inhibitor, such as tranexamic acid, epsilon-amino-caproic acid, or a combination thereof. In certain embodiments, the clotting agent comprises tranexamic acid.

In some embodiments, the clotting agent can be present in the synthetic whole blood substitute in an amount of from 1 mg/mL to 15 mg/mL, such as about 10 mg/mL.

### Plasma Substitutes and Plasma Expanders

The synthetic whole blood substitute can include a plasma substitute, a plasma expander, or a combination thereof. Examples of suitable plasma substitutes and/or plasma expanders include polymerized albumin, polymer conjugated polymerized albumin, PEG conjugated albumin, lactated Ringers, saline, human plasma, or a combination thereof.

The plasma substitute, a plasma expander, or a combination thereof can be present in the blood substitute in an amount of from 15 mg/mL to 75 mg/mL, such as from 20 mg/mL to 60 mg/mL.

In some embodiments, the plasma expander can include but is not limited to human serum albumin.

In some embodiments, the plasma substitute, plasma expander, or combination thereof can comprise polymerized albumin (e.g., polymerized human serum albumin) and/or polymer-modified polymerized albumin (e.g., PEGylated polymerized human serum albumin) and/or polymer-modified albumin.

The albumin can comprise any albumin known to those of skill in the art. In some embodiments, the albumin may be serum albumin isolated from a host species and purified for use in the formation of a conjugate. The serum albumin may be any mammalian serum albumin known to those of skill in the art, including but not limited to mouse, rat, rabbit, guinea pig, dog, cat, sheep, bovine, ovine, equine, or human albumin. In some embodiments, the albumin is human serum albumin (HSA).

In some cases, the albumin can be obtained from serum or a genomic source. In other cases, the albumin can comprise recombinant albumin. The recombinant albumin may be any mammalian albumin known to those of skill in the art, including but not limited to mouse, rat, rabbit, guinea pig, dog, cat, sheep, bovine, ovine, equine, or human albumin. In some cases, the recombinant albumin is recombinant human albumin, in particular, recombinant human serum albumin (rHSA).

Human serum albumin (HSA) is responsible for a significant proportion of the osmotic pressure of serum and also functions as a carrier of endogenous and exogenous ligands. In its mature form, HSA is a non-glycosylated monomeric protein of 585 amino acids, corresponding to a molecular weight of about 66 kDa. Its globular structure is maintained by 17 disulfide bridges which create a sequential series of 9 double loops.

In some embodiments, the composition can include polymerized albumin, wherein the polymerized albumin includes less than 5% by weight low molecular weight albumin species having a molecular weight less than 100 kDa and less than 5% by weight high molecular weight albumin species.

In some embodiments, the polymerized albumin includes less than 5% by weight low molecular weight albumin species having a molecular weight less than 100 kDa and less than 5% by weight high molecular weight albumin species, such as less than 1% by weight low molecular weight albumin species having a molecular weight less than 100 kDa and less than 1% by weight high molecular weight albumin species. In some embodiments, the low molecular weight albumin species include unreacted albumin, albumin oligomers, or combinations thereof.

In some embodiments, the high molecular weight albumin species comprise albumin species that are retained on a filtration membrane having a pore size of 0.2 μm.

In some embodiments, the high molecular weight albumin species comprise albumin species having a molecular weight greater than 10,000 kDa.

In some embodiments, the high molecular weight albumin species comprise albumin species having a molecular weight greater than 2000 kDa.

In some embodiments, the polymerized albumin comprises less than 5% by weight albumin species having a molecular weight less than 100 kDa; and less than 5% by weight albumin species that pass through a filtration membrane having a pore size of 0.2 μm.

In some embodiments, the polymerized albumin comprises less than 5% by weight albumin species having a molecular weight less than 100 kDa; and less than 5% by weight albumin species having a molecular weight greater than 10,000 kDa.

In some embodiments, the polymerized albumin comprises less than 5% by weight albumin species having a molecular weight less than 100 kDa; and less than 5% by weight albumin species having a molecular weight greater than 500 kDa.

In some embodiments, the polymerized albumin comprises less than 5% by weight albumin species having a molecular weight less than 200 kDa; and less than 5% by weight albumin species that pass through a filtration membrane having a pore size of 0.2 μm.

In some embodiments, the polymerized albumin comprises less than 1% by weight albumin species having a molecular weight less than 100 kDa; and less than 1% by weight albumin species that pass through a filtration membrane having a pore size of 0.2 μm.

In some embodiments, the polymerized albumin comprises less than 1% by weight albumin species having a molecular weight less than 100 kDa; and less than 1% by weight albumin species having a molecular weight greater than 10,000 kDa.

In some embodiments, the polymerized albumin comprises less than 1% by weight albumin species having a molecular weight less than 100 kDa; and less than 1% by weight albumin species having a molecular weight greater than 2000 kDa.

In some embodiments, the polymerized albumin comprises less than 1% by weight albumin species having a molecular weight less than 500 kDa; and less than 1% by weight albumin species that pass through a filtration membrane having a pore size of 0.2 μm.

In some embodiments, the polymerized albumin is prepared by a process that includes: polymerizing albumin; filtering the polymerized albumin by ultrafiltration against a first filtration membrane having a pore size that separates the low molecular weight albumin species from the polymerized albumin; and filtering the polymerized albumin by ultrafiltration against a second filtration membrane having a pore size that separates the high molecular weight albumin species from the polymerized albumin.

As used herein, “filtration membrane” refers to microporous barriers of, for example, polymeric, ceramic, or metallic materials which are used to separate dissolved materials (solutes), colloids, or find particular from solutions. A filtration membrane can be rated for retaining solutes have a specific molecular weight range from the molecular weight of one component in the solution to another component in the solution. By way of example, a filtration membrane can be rated for retaining polymerized albumin with a molecular weight above that of a low molecular weight albumin species (e.g., such as a membrane rated for retaining solutes have a molecular weight above 500 kDa). A filtration membrane can also be rated based on its pore size (e.g., a pore size of 0.2 μm).

In some embodiments, the first filtration membrane can be rated for retaining solutes having a molecular weight greater than the molecular weight of the low molecular weight albumin species, thereby forming a retentate fraction including the polymerized albumin and a permeate fraction including the low molecular weight albumin species. As used herein, a “retentate fraction” refers to the fraction of solution that is unable to pass through the filtration membrane. In some embodiments, the retentate fraction can include polymerized albumin. As used herein, a “permeate fraction” refers to the fraction of solution that permeates the filtration membrane.

In some embodiments, ultrafiltration can include tangential-flow filtration. Tangential-flow filtration refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (e.g., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

In some embodiments, the first filtration membrane is rated for retaining solutes having a molecular weight greater than 100 kDa, thereby forming a retentate fraction comprising the polymerized albumin and a permeate fraction comprising low molecular weight albumin species having a molecular weight less than 100 kDa. In some embodiments, the second filtration membrane is rated for retaining solutes having a molecular weight greater than 500 kDa, thereby forming a retentate fraction comprising the high molecular weight albumin species and a permeate fraction comprising the polymerized albumin. In other embodiments, the second filtration membrane has a pore size of 0.2 μm, thereby forming a retentate fraction comprising the high molecular weight albumin species and a permeate fraction comprising the polymerized albumin.

In some embodiments, the first filtration membrane is rated for retaining solutes having a molecular weight greater than 500 kDa, thereby forming a retentate fraction comprising the polymerized albumin and a permeate fraction comprising low molecular weight albumin species having a molecular weight less than 500 kDa. In some embodiments, the second filtration membrane has a pore size of 0.2 μm, thereby forming a retentate fraction comprising the high molecular weight albumin species and a permeate fraction comprising the polymerized albumin.

In some embodiments, the polymerized albumin can have a cross-linker to albumin molar ratio, referred to herein as the cross-link density, of at least 10:1, (e.g., at least 20:1, at least 30:1, at least 40:1, at least 50:1, at least 60:1, at least 70:1, at least 80:1, at least 90:1). In some embodiments, the polymerized albumin can have a cross-linker to albumin molar ratio, referred to herein as the cross-link density, of 100:1 or less, (e.g., 90:1 or less, 80:1 or less, 70:1 or less, 60:1 or less, 50:1 or less, 40:1 or less, 30:1 or less, 20:1 or less), and may optionally range between any of these cross-linking densities.

The polymerized albumin can have a cross-linker to albumin molar ratio, referred to herein as the cross-link density, ranging from any of the minimum values described above to any of the maximum values described above. For example, the cross-linking density can be in a range from 10:1 to 100:1 (e.g., from 10:1 to 20:1, from 10:1 to 30:1, from 10:1 to 40:1, from 10:1 to 50:1, from 10:1 to 60:1, from 10:1 to 70:1, from 10:1 to 80:1, from 10:1 to 90:1, from 20:1 to 30:1, from 20:1 to 40:1, from 20:1 to 50:1, from 20:1 to 60:1, from 20:1 to 70:1, from 20:1 to 80:1, from 20:1 to 90:1, from 20:1 to 100:1, from 30:1 to 40:1, from 30:1 to 50:1, from 30:1 to 60:1, from 30:1 to 70:1, from 30:1 to 80:1, from 30:1 to 90:1, from 30:1 to 100:1, from 40:1 to 50:1, from 40:1 to 60:1, from 40:1 to 70:1, from 40:1 to 80:1, from 40:1 to 90:1, from 40:1 to 100:1, from 50:1 to 60:1, from 50:1 to 70:1, from 50:1 to 80:1, from 50:1 to 90:1, from 50:1 to 100:1, 60:1 to 70:1, from 60:1 to 80:1, from 60:1 to 90:1, from 60:1 to 100:1, from 70:1 to 80:1, from 70:1 to 90:1, from 70:1 to 100:1, from 80:1 to 90:1, from 80:1 to 100:1, from 90:1 to 100:1). The molecular weight and/or cross-link density of the polymerized albumin compositions affect their biophysical characteristics, which directly determine viscosity and colloid osmotic pressure. As shown in the examples below, high MW polymerized albumin compositions having higher cross-link densities generally have improved biophysical characteristics relative to native albumin and dextran.

The polymerized albumin can have a higher viscosity than monomeric albumin compositions, when formulated at the same protein concentration. In one embodiment, the viscosity of the polymerized albumin composition is 1.1 times greater than the viscosity of the monomeric albumin composition having the same concentration (e.g., 2 times greater, 3 times greater, 4 times greater, 5 times greater, 6 times greater, 7 times greater, 8 times greater, 9 times greater, or 10 times greater).

In some embodiments, the polymerized albumin compositions have a lower COP than monomeric compositions at the same concentration level. In one embodiment, the COP of the polymerized albumin composition can be ½ the COP of monomeric albumin (e.g., ⅓, ¼, ⅕, ⅙, 1/7, ⅛, 1/9, 1/10, 1/15, 1/20, 1/25, 1/30, 1/35, 1/40, 1/45, or 1/50 the COP of monomeric albumin). In another embodiment, the COP of the polymerized albumin composition is about 1/10 the COP of monomeric albumin. In another embodiment, the COP is about 1/50 the COP of monomeric albumin.

In some embodiments, the polymerized albumin may have a molecular weight of at least 130 kDa and a cross-link density of at least 10:1. In some embodiments, the polymerized albumin may have a molecular weight of at least 200 kDa and a cross-link density of at least 10:1. In some embodiments, the polymerized albumin may have a molecular weight of at least 300 kDa and a cross-link density of at least 10:1. In some embodiments, the polymerized albumin may have a molecular weight of at least 400 kDa and a cross-link density of at least 10:1. In some embodiments, the polymerized albumin may have a molecular weight of at least 500 kDa and a cross-link density of at least 10:1. In some embodiments, the polymerized albumin may have a molecular weight of at least 130 kDa and a cross-link density of at least 25:1. In some embodiments, the polymerized albumin may have a molecular weight of at least 200 kDa and a cross-link density of at least 25:1. In some embodiments, the polymerized albumin may have a molecular weight of at least 300 kDa and a cross-link density of at least 25:1. In some embodiments, the polymerized albumin may have a molecular weight of at least 400 kDa and a cross-link density of at least 25:1. In some embodiments, the polymerized albumin may have a molecular weight of at least 500 kDa and a cross-link density of at least 25:1. In some embodiments, the polymerized albumin may have a molecular weight of at least 130 kDa and a cross-link density of at least 50:1. In some embodiments, the polymerized albumin may have a molecular weight of at least 200 kDa and a cross-link density of at least about 50:1. In some embodiments, the polymerized albumin may have a molecular weight of at least 300 kDa and a cross-link density of at least 50:1. In some embodiments, the polymerized albumin may have a molecular weight of at least 400 kDa and a cross-link density of at least 50:1. In some embodiments, the polymerized albumin may have a molecular weight of at least 500 kDa and a cross-link density of at least 50:1. In some embodiments, the polymerized albumin may have a molecular weight of at least 130 kDa and a cross-link density of at least 75:1. In some embodiments, the polymerized albumin may have a molecular weight of at least 200 kDa and a cross-link density of at least 75:1. In some embodiments, the polymerized albumin may have a molecular weight of at least 300 kDa and a cross-link density of at least 75:1. In some embodiments, the polymerized albumin may have a molecular weight of at least 400 kDa and a cross-link density of at least 75:1. In some embodiments, the polymerized albumin may have a molecular weight of at least 500 kDa and a cross-link density of at least 75:1. In some embodiments, the polymerized albumin may have a molecular weight of at least 130 kDa and a cross-link density of at least 100:1. In some embodiments, the polymerized albumin may have a molecular weight of at least 200 kDa and a cross-link density of at least 100:1. In some embodiments, the polymerized albumin may have a molecular weight of at least about 300 kDa and a cross-link density of at least 100:1. In some embodiments, the polymerized albumin may have a molecular weight of at least 400 kDa and a cross-link density of at least 100:1. In some embodiments, the polymerized albumin may have a molecular weight of at least 500 kDa and a cross-link density of at least 100:1.

In some embodiments, the polymerized albumin can be made by polymerizing monomeric albumin with a cross-linker, quenching the polymerization reaction with a reducing agent, and collecting the polymerized albumin having the desired molecular weight.

As discussed above, suitable monomeric albumin can come from any source such as human serum albumin isolated from human serum using known techniques or recombinant human serum albumin. The monomeric albumin is diluted or concentrated to the desired level, such as to 25 mg/mL with a suitable buffer. The polymerization reaction is initiated by the addition of a cross-linker, such as a 70% glutaraldehyde solution, to the polymerized albumin solution at the desired molar ratio of cross-linker to albumin: such as at least 10:1, at least 50:1, and at least 100:1. The cross-linking density of the resulting polymerized albumin composition may be controlled by controlling this molar ratio or by controlling the parameters of the polymerization reaction, such as the duration and temperature of the reaction. The cross-link density of a polymerized albumin composition can be confirmed by separating polymerized albumin from any free cross-linker after the polymerization reaction and quantifying the amount of free cross-linker compared to the initial amount of cross-linker used in the reaction. The difference between the two quantities would be equivalent to the amount of cross-linker that is cross-linked to the protein. Glutaraldehyde, like many cross-linkers, reacts with lysine, histidine, tyrosine, arginine, and primary amine groups, forming both intra and intermolecular cross-links within albumin and between neighboring albumin molecules in solution. Therefore, cross-linked albumin compositions can include polymers of various molecular weights.

Suitable cross-linkers in addition to glutaraldehyde can include succindialdehyde, activated forms of polyoxyethylene and dextran, α-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and compounds belonging to the bisimidate class, the acyl diazide class or the aryl dihalide class, and combinations thereof.

The albumin is allowed to polymerize with the cross-linker for a suitable period of time to obtain polymerized albumin having the desired MW. For example, the polymerization reaction may be incubated at about 37° C. for between 1 and 4 hours. The polymerization reaction is then quenched with a molar excess of reducing agent, preferably a strong reducing agent that is capable of reducing the Schiff bases in the polymerized albumin and any remaining free aldehyde groups on the cross-linker. For example, the reaction may be quenched by incubating the reaction mixture with a 1 M sodium borohydride solution for 30 min at 37° C. Quenching the Schiff bases in the polymerized albumin stabilizes the polymer and prevents the hydrolysis of polymerized albumin back to monomeric albumin, which could extravasate and cause detrimental side effects. Moreover, reducing the aldehyde group on the cross-linker completely quenches the polymerization reaction. An exemplary strong reducing agent capable for use in embodiments of the invention is sodium borohydride, however it is understood that other reducing agents may be useful as well.

The MW distribution of the polymerized albumin in the quenched reaction mixture will be affected by the conditions under which the polymerization reaction is conducted, such as duration and temperature of the incubation along with the cross-linker to albumin molar ratio. To control for variables in the polymerization reaction that might result in polymerized albumin having a MW outside of the desired range, the process further includes the step of collecting polymerized albumin having the desired MW range. The collecting step may include separating or purifying polymerized albumin having the desired MW range or making the polymerized albumin free from undesirable elements such as albumin having a MW outside of the desired range. For example, the polymerized albumin solution may be clarified such as by being passed through a glass chromatography column packed with glass wool to remove large particles. The clarified polymerized albumin solution is then separated into distinct molecular mass fractions using known separation methods such as passing the clarified polymerized albumin solution through a tangential flow filtration (TFF) hollow fiber (HF) cartridge selected to collect polymerized albumin having the desired MW. For example, fractionation of the polymerized albumin composition with a 100 kDa TFF HF cartridge (Spectrum Labs, Rancho Dominguez, Calif.) will result in the retentate containing polymerized albumin molecules that are at least 100 kDa or larger and that fall within the desired MW in one embodiment. In that example, the filtrate will mostly contain polymerized albumin molecules that are smaller than 100 kDa, i.e., molecules that are smaller than the desired MW. The MW of the polymerized albumin can be controlled by passing the clarified polymerized albumin solution through TFF HF cartridges having different pore sizes selective for the desired MW.

The polymerized albumin solution may then be subjected to as many cycles of diafiltration with an appropriate buffer as needed in order to remove impurities having a MW outside of the desired range. The polymerized albumin solution may also buffer exchanged to remove impurities such as unpolymerized cross-linkers and quenching agents which may be cytotoxic. After separation of the desired fraction, the filtrate may subsequently be concentrated such as with a 100 kDa TFF HF cartridge (Spectrum Labs). The MW distribution of the polymerized albumin may be confirmed by known methods such as SDS-PAGE analysis or size exclusion chromatography coupled with multi-angle static light scattering.

In some embodiments, the polymerized albumin is covalently modified with a polymer. In some embodiments, the polymer comprises a polyalkylene oxide, such as polyethylene glycol (PEG). In other embodiments, the polymer comprises a zwitterionic polymer. In some embodiments, the polymer has a molecular weight of from 200 Da to 1,000,000 Da, such as from 1,000 Da to 10,000 Da, or from 3,000 Da to 6,000 Da, or about 5,000 Da.

In some embodiments, the polymerized albumin is covalently modified with the polymer in a process that comprises: conjugating one or more polymers to the polymerized albumin to form a polymer-modified polymerized albumin; filtering the polymer-modified polymerized albumin by ultrafiltration against a first filtration membrane having a pore size that separates low molecular weight contaminants from the polymer-modified polymerized albumin; and filtering the polymer-modified polymerized albumin by ultrafiltration against a second filtration membrane having a pore size that separates the high molecular weight contaminants from the polymer-modified polymerized albumin. In some embodiments, the ultrafiltration comprises tangential-flow filtration.

In some embodiments, the first filtration membrane is rated for retaining solutes having a molecular weight greater than 100 kDa, thereby forming a retentate fraction comprising the polymer-modified polymerized albumin and a permeate fraction comprising low molecular weight contaminants having a molecular weight less than 100 kDa. In some embodiments, the second filtration membrane is rated for retaining solutes having a molecular weight greater than 500 kDa, thereby forming a retentate fraction comprising the high molecular weight contaminants and a permeate fraction comprising the polymer-modified polymerized albumin. In other embodiments, the second filtration membrane has a pore size of 0.2 μm, thereby forming a retentate fraction comprising the high molecular weight contaminants and a permeate fraction comprising the polymer-modified polymerized albumin.

In some embodiments, the first filtration membrane is rated for retaining solutes having a molecular weight greater than 500 kDa, thereby forming a retentate fraction comprising the polymer-modified polymerized albumin and a permeate fraction comprising low molecular weight contaminants having a molecular weight less than 500 kDa. In some embodiments, the second filtration membrane has a pore size of 0.2 μm, thereby forming a retentate fraction comprising the high molecular weight albumin species and a permeate fraction comprising the polymerized albumin.

In certain embodiments, the polymerized albumin is covalently modified with a polyethylene glycol (PEG) polymer. In some embodiments, the PEG polymer can have a molecular weight of from 200 Da to 20,000 Da, such as from 1,000 Da to 10,000 Da, or from 3,000 Da to 6,000 Da, or about 5,000 Da.

In some embodiments, the polymerized albumin is covalently modified with the PEG polymer in a process that includes: conjugating one or more PEG polymers to the polymerized albumin to form PEGylated polymerized albumin; filtering the PEGylated polymerized albumin by ultrafiltration against a first filtration membrane having a pore size that separates low molecular weight contaminants from the PEGylated polymerized albumin; and filtering the PEGylated polymerized albumin by ultrafiltration against a second filtration membrane having a pore size that separates the high molecular weight contaminants from the PEGylated polymerized albumin.

In some embodiments, conjugating the one or more polyethylene glycol (PEG) polymers to the polymerized albumin to form the PEGylated polymerized albumin can comprise contacting the polymerized albumin with a derivatized PEG under conditions permitting formation of a covalent bond between the PEG and the polymerized albumin so as to form the PEGylated polymerized albumin. In some examples, the derivatized PEG can comprise succinimidyl-PEG, cyanuric chloride-PEG, or maleimide-PEG. In certain examples, conjugating the one or more polyethylene glycol (PEG) polymers to the polymerized albumin to form the PEGylated polymerized albumin can further comprise contacting the PolyHSA with a thiolation reagent, such as 2-iminothiolane hydrochloride, to introduce thiol moieties that react with the derivatized PEG.

### Methods of Use

The compositions described herein can be used as blood substitutes or additives to blood or other solutions. As such, these compositions can be administered to subjects suffering with a wide range of diseases, disorders, and conditions.

The compositions described herein can exhibit reversible oxygen binding capacities which provide for oxygen transport properties. The compositions described herein can demonstrate good loading and unloading characteristics in usage which can correlate to having an oxygen-hemoglobin dissociation curve (P₅₀) similar to whole blood. The HBOC compositions described herein can show a high affinity for binding oxygen in the capillaries through the lungs and then adequately release oxygen to the tissues in the body.

Insofar as the physiological properties are concerned, the compositions described herein can not cause vasoconstriction, renal toxicity, hemoglobinurea and other problems implicated with intravenous administration of known oxygen carriers. Upon intravenous administration of the compositions described herein, no appreciable decrease in urine production, no appreciable decrease in glomerular filtration rate, no appreciable extravasation into the peritoneal cavity and/or no appreciable change in the color of urine produced can be observed in the subject.

In some embodiments, the compositions described herein can find application in the treatment of trauma, myocardial infarction, stroke, acute anemia and oxygen deficiency disorders such as hypoxemia, hypoxia or end stage hypoxia due to impairment or failure of the lung to fully oxygenate blood. The compositions described herein can also be used to diseases or medical conditions requiring a resuscitative fluid (e.g., trauma, specifically hemorrhagic shock), intravascular volume expander or exchange transfusion. In addition to medical treatment, the compositions described herein can also be used to preserve organs for transplantation.

In some cases, the compositions described herein can be administered to a subject to treat a loss of blood due to injury, hemolytic anemia, equine infectious anemia, feline infectious anemia, a bacterial infection, Factor IV fragmentation, hypersplenation, splenomegaly, hemorrhagic syndrome, hypoplastic anemia, aplastic anemia, idiopathic immune hemadytic conditions, iron deficiency, isoimmune hemdytic anemia, microangiopathic hemolytic anemia, parasitism, or any combination thereof.

The compositions described herein can also be used in a variety of applications where a rapid restoration of O₂ levels or an increased O₂ level or a replacement of O₂ levels is clinically indicated, such as the following:

Trauma. An acute loss of whole blood can result in a fluid shift from the interstitial and intracellular spaces to replace the lost volume of blood while shunting of blood away from the low priority organs including the skin and gut. Shunting of blood away from organs reduces and sometimes eliminates O₂ levels in these organs and results in progressive tissue death. Rapid restoration of O₂ levels is contemplated as perhaps resulting in a significantly better salvage of tissues in patients suffering such acute blood loss.

Ischemia. In ischemia, a particular organ (or organs) is “starved” for oxygen. Small sections of the organ, known as infarcts, begin to die as a result of the lack of O₂. Rapid restoration of O₂ levels is critical is stemming infarct formation in critical tissues. Conditions resulting in ischemia include heart attack, stroke, or cerbrovascular trauma.

Hemodilution: In this clinical application, a blood substitute is required to replace blood that is removed pre-operatively. It is contemplated that the patient blood removal occurs to prevent a requirement for allogeneic transfusions post-operatively. In this application, the blood substitute is administered to replace (or substitute for) the O₂ levels of the removed autologous blood. This permits the use of the removed autologous blood for necessary transfusions during and after surgery. One such surgery requiring pre-operative blood removal would be a cardiopulmonary bypass procedure.

Septic Shock. In overwhelming sepsis, some patients may become hypertensive in spite of massive fluid therapy and treatment with vasocontrictor agents. In this instance, the overproduction of nitric oxide (NO) results in the lowered blood pressure. Therefore, hemoglobin is close to an ideal agent for treatment of these patients because hemoglobin binds NO with an avidity that parallels O₂.

Cancer. Delivery of O₂ to the hypoxic inner core of a tumor mass increases its sensitivity to radiotherapy and chemotherapy. Because the microvasculature of a tumor is unlike that of other tissues, sensitization through increasing O₂ levels requires O₂ be unloaded within the hypoxic core. In other words, the P₅₀ should be very low to prevent early unloading of the O₂, increasing the O₂ levels, to insure optimal sensitization of the tumor to subsequent radiation and chemotherapy treatments.

Chronic anemia. In these patients, replacement of lost or metabolized hemoglobin is compromised or completely absent. It is contemplated that the blood substitute must effectively replace or increase the reduced O₂ levels in the patient.

Sickle cell anemia. In sickle cell anemia, the patient is debilitated by a loss of O₂ levels that occurs during the sickling process as well as a very high red blood cell turnover rate. The sickling process is a function of P_{O2} where the lower the P_{O2}, the greater the sickling rate. It is contemplated that the ideal blood substitute would restore patient O₂ levels to within a normal range during a sickling crisis.

Cardioplegia. In certain cardiac surgical procedures, the heart is stopped by appropriate electrocyte solutions and reducing patient temperature. Reduction of the temperature will significantly reduce the P₅₀, possibly preventing unloading of O₂ under any ordinary physiological conditions. Replacement of O₂ levels is contemplated as potentially reducing tissue damage and death during such procedures.

Hypoxia. Soldiers, altitude dwellers, and world-class athletes under extreme conditions may suffer reduced O₂ levels because extraction of O₂ from air in the lung is limited. The limited O₂ extraction further limits O₂ transport. It is contemplated that a blood substitute could replace or increase the O₂ levels in such individuals.

Organ Perfusion. During the time an organ is maintained ex vivo, maintaining O₂ content is essential to preserving structural and cellular integrity and minimizing infarct formation. It is contemplated that a blood substitute would sustain the O₂ requirements for such an organ.

Cell Culture. This requirement is virtually identical to that of organ perfusion, except that the rate of O₂ consumption may be higher.

Hematopoiesis. It is contemplated that the blood substitute serves as a source for heme and iron for use in the synthesis of new hemoglobin during hematopoiesis.

The compositions described herein can also be used in non-humans, including domestic animals such as livestock and companion animals (e.g, dogs, cats, horses, birds, reptiles), as well as other animals in aquaria, zoos, oceanaria, and other facilities that house animals.

### EXAMPLES

The invention will be described in greater detail by way of specific examples. The following examples are offered for illustrative purposes and are not intended to limit the invention in any manner. Those of skill in the art will readily recognize a variety of non-critical parameters which can be changed or modified to yield essentially the same results.

### Example 1. Photocatalytic Synthesis of a Polydopamine-Coated Acellular Mega-Hemoglobin as a Potential Oxygen Therapeutic with Antioxidant Properties

### Overview

Hemoglobin-based oxygen carriers (HBOCs) are being developed to overcome limitations associated with transfusion of donated red blood cells (RBCs) such as potential transmission of blood borne pathogens and limited ex vivo storage shelf-life. Annelid erythrocruorin (Ec) derived from the worm Lumbricus terrestris (Lt) is an acellular mega-hemoglobin that has promise as a potential HBOC due to the large size of its oligomeric structure, thus overcoming limitations of unmodified circulating cell-free hemoglobin (Hb). With a large molecular weight of 3.6 MDa compared to 64.5 kDa for human Hb (hHb) and 144 oxygen-binding globin subunits compared to the 4 globin subunits of hHb, LtEc does not extravasate from the circulation to the same extent as hHb. LtEc is stable in the circulation without RBC membrane encapsulation, and has a lower rate of auto-oxidation compared to acellular hHb, which allows the protein to remain functional for longer periods of time in the circulation compared to HBOCs derived from mammalian Hbs.

Surface coatings, such as poly-ethylene glycol (PEG) and oxidized dextran (Odex), have been investigated to potentially reduce the immune response and improve the circulation time of LtEc in vivo. Polydopamine (PDA) is a hydrophilic, biocompatible, bioinspired polymer coating used for biomedical nanoparticle assemblies and coatings and has been investigated for the surface-coating of hHb. PDA is typically synthesized via the self-polymerization of dopamine (DA) under alkaline (pH >8.0) conditions. However, at pH >8.0, the oligomeric structure of LtEc begins to dissociate. Therefore, in this example, we investigated a photocatalytic method of PDA polymerization on the surface of LtEc using 9-mesityl-10-methylacridinium tetrafluoroborate (Acr-Mes) to drive PDA polymerization under physiological conditions (pH 7.4, 25° C.) over 2, 5, and 16 hours in order to preserve the size and structure of LtEc. The resulting structural, biophysical, and antioxidant properties of PDA surface coated LtEc (PDA-LtEc) was characterized using various techniques. PDA-LtEc showed an increase in measured particle size, molecular weight, and surface ζ-potential with increasing reaction time from t=2 hours to t=16 hours compared to unmodified LtEc. PDA-LtEc reacted for 16 hours was found to have reduced oxygen-binding cooperativity and slower deoxygenation kinetics compared to PDA-LtEc with lower levels of polymerization (t=2 hours), but there was no statistically significant difference in oxygen affinity. The thickness of the PDA coating can be controlled and in turn the biophysical properties can be tuned by changing various reaction conditions. PDA-LtEc was shown to demonstrate an increased level of antioxidant capacity (ferric iron reduction and free radical scavenging) when synthesized at a reaction time of t=16 hours compared to LtEc. These antioxidant properties may prove beneficial for oxidative protection of PDA-LtEc during its time in the circulation. Hence, we believe that PDA-LtEc is a promising oxygen therapeutic for potential use in transfusion medicine applications.

### Materials and Methods

Materials. Canadian Nightcrawlers (Lumbricus terrestris) were purchased from Wholesale Bait Company (Hamilton, OH) and kept at 4° C. before purification of LtEc. Hydrochloric acid (HCl), sodium phosphate monobasic (NaH₂PO₄), and sodium phosphate dibasic (Na₂HPO₄) were obtained from Fisher Scientific (Hampton, NH). 2,4,6 tripyridyl-S-triazine (TPTZ) was obtained from TCI Chemicals (Portland, OR). 9-mesityl-10-methylacridinium tetrafluoroborate (Acr-Mes), dopamine HCl (C₈H₁₂ClNO₂), FeCl₃, ascorbic acid (C₆H₈O₆), 2,2′-azinobis-(3-ethylbenzothiazoline-6-sulfonic acid) (ABTS), and all other chemical reagents were purchased from Sigma-Aldrich (St. Louis, MO). All hollow fiber (HF) tangential flow filtration (TFF) modules were purchased from Repligen (Waltham, MA).

Earthworm Preparation and LtEc Purifications. LtEc was prepared using methods described by Savla, et. al. Briefly, one thousand earthworms were blended in a Tris-EDTA buffer solution pH 7.0. LtEc was crudely separated from the earthworm remnants by a two-step centrifugation process: first for 40 minutes at 4° C. at 3,700 g and again for another 50 minutes at 10° C. and 18,000 g. The final stage before LtEc purification was a vacuum filtration step at 4° C. with a 4-6 m filter.

LtEc was purified via a scalable multi-stage tangential flow filtration (TFF) process. To remove larger particulates, the crude LtEc was filtered using a 0.5 μm polyethersulfone (PES) HF filter in stage 1. In stage 2, the product from stage 1 was filtered using a 0.2 μm PES HF filter to remove particles larger than the oligomeric structure of LtEc and other bacterial components. In stage 3, the permeated protein mixture was subjected to multiple continuous diafiltration cycles over a 500 kDa PS HF filter using PBS or modified lactated Ringer's solution at pH 7.4 for ~30 diacycles. The purified LtEc was concentrated down to >100 mg/mL and stored at −80° C. and thawed before use.

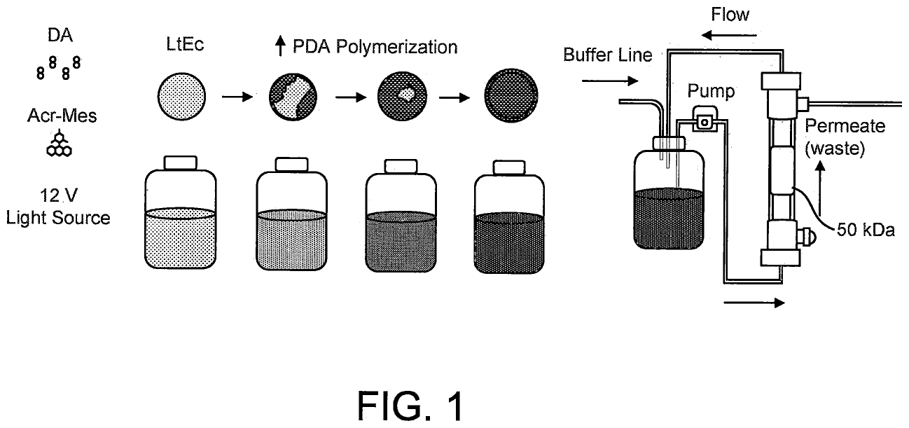

PDA-LtEc Synthesis. LtEc was diluted to a concentration of 3.5 mg/mL (0.14 μL) and reacted with dopamine HCl in excess (2.25 mg/mL, 100:1 per-heme ratio) and the photocatalyst Acr-Mes to 0.125 mg/mL (800:1 DA to Acr-Mes molar ratio) in PBS buffer, pH 7.4. The reaction was driven by a 12 V light source and different species of PDA-LtEc were synthesized with 2-hour, 5-hour, and 16-hour reactions at 25° C. (FIG. 1). The reaction was quenched with the addition of ascorbic acid (0.75 mg/mL), and unreacted substrates were removed by TFF with 10 diafiltrations of PBS using a 50 kDa HF filter. The PDA-LtEc batches were then stored at −80° C. and thawed before use.

Structural Analysis. Relative changes in protein MW were measured with size exclusion high-performance liquid chromatography (SEC-HPLC). LtEc and PDA-LtEc samples were prepared by dilution to a concentration of 1 mg/mL in PBS and sterile filtered with a 0.2 μm syringe filter before injection into the column. The samples were separated using a Dionex UltiMate 3000 UHPLC/HPLC system (Thermo Fisher Scientific, Waltham, MA) with an analytical Acclaim SEC-1000 (4.6×300 mm) column (Thermo Fisher Scientific, Waltham, MA) at a flow rate of 0.35 mL/min. Elution peaks were monitored by UV-visible absorption at 280 nm (protein) and 413 nm (heme).

The thickness of the PDA coating and resulting particle size changes were measured using dynamic light scattering (DLS). Protein samples for DLS analysis were prepared by dilution to 1 mg/mL in deionized (DI) water and light scattering was measured at a wavelength of 637 nm and an angle of 900 with a BI-200SM goniometer (Brookhaven Instruments Corp., Holtsville, NY). Diluted ~1 mg/mL protein samples were subsequently used to measure changes in surface zeta potential using Brookhaven Instruments ZetaPals (Holtsville, NY).

Oxygen Equilibrium and Dissociation Kinetics. Samples were diluted to ~1 mg/mL in 5 mL of Hemox buffer (pH 7.4) for measurement of oxygen equilibrium curves with a Hemox Analyzer (TCS Scientific Corp., New Hope, PA). 20 μL of additive A and 10 μL of additive B and 20 μL of anti-foaming agent (TCS Scientific) were added, and the samples were maintained at 37.0±0.1° C. for each run. Samples were first saturated with oxygen, then the absorbances of oxy- and deoxy-Hb were measured as the samples were flushed with nitrogen. The P₅₀ (partial pressure of oxygen at which Hb is half saturated with oxygen) and cooperativity coefficient were regressed by fitting the curves to the Hill equation.

Rapid deoxygenation kinetics were measured using stopped-flow UV-visible spectrophotometry. Deoxygenated buffer was prepared by the addition of 1.5 mg/mL sodium dithionite to PBS and bubbling under nitrogen gas for 30 minutes. Oxygenated protein samples were diluted to a concentration of 12.5 μM (per heme basis) for analysis. A microvolume stopped-flow spectrophotometer (Applied Photophysics Ltd., Surrey, U.K.) was used to mix the deoxygenated buffer and protein samples. The change in absorbance at 437.5 nm was measured in order to monitor the oxygen offloading kinetics of LtEc and PDA-LtEc. The kinetics were fit to an exponential decay function to regress the oxygen offloading rate constant (k_{off},O₂) for LtEc and different species of PDA-LtEc.

Antioxidant Assays. The ferric reducing/antioxidant power (FRAP) and ABTS cation decolorization assays were used to assess the antioxidant capabilities of PDA-LtEc compared to that of native LtEc. The FRAP assay was adapted from a method that assessed the antioxidant capacity of PDA-Hb. FRAP reagent solution was prepared by mixing acetate buffer (300 mM, pH 3.6), TPTZ solution (10 mM TPTZ in 40 mM HCl), and FeCl3 (20 mM) in a 10:1:1 ratio by volume. The TPTZ acts as an oxidizing agent that ensures that aqueous Fe remains in the ferric Fe³⁺ state. The FRAP reagent was warmed to 37° C. and protein samples were diluted to 5 mg/mL. Reference solutions of FRAP reagent in PBS (FRAP reference) and 5 mg/mL of Trolox in FRAP reagent were also prepared.

Samples were added to the FRAP solution in a 1:20 ratio (10 μL sample to 200 μL reagent) and incubated at 37° C. for 4 min. The change in absorbance values at 593 nm were measured by plate reader (extinction coefficient 21,140 M⁻¹ cm⁻¹). The change in absorbance from the control was used to compare the relative Fe³⁺ reducing ability of the protein samples (Equation 1).

$$Change in Absorbance = {Abs}_{FRAP + sample} = {Abs}_{FRAP} \qquad Equation 1$$

An ABTS decolorization assay was similarly adapted from the literature to assess the free radical scavenging antioxidant capability of PDA-LtEc. ABTS stock solution with oxidized ABTS^{·+} free radicals was prepared by mixing 7 mM ABTS and sodium periodate (NaIO₄, 4.90 mM) and stored in the dark. The ABTS stock concentration was adjusted until the plate reader absorbance of the stock solution read ~0.7 mAU. Protein samples were diluted to 5 mg/mL, added to the ABTS solution in a 1:20 ratio (10 μL sample to 200 μL reagent), and reacted in the dark for 5 min at 20° C. An ABTS reference solution of PBS in ABTS stock solution stock (1:20 volume ratio) was also prepared. Samples were measured by plate reader for the change in absorbance at 734 nm compared to baseline (extinction coefficient 1.5×10⁴ M⁻¹ cm⁻¹). The free radical scavenging ratios of the samples were determined by the reduction of ABTS^{·+} to ABTS²⁻, which was monitored by the absorbance decrease at 734 nm (Equation 2).

$$Scavenging ratio = \frac{{Abs}_{ABTS} - ( {Abs}_{ABTS + Sample} - {Abs}_{Sample} )}{{Abs}_{ABTS}} \times 100 \qquad Equation 2$$

Statistical Analysis. Measurements of PDA-LtEc samples were performed in triplicate on separate batches of material. Statistical analysis was performed using JMP Pro 15 software. ANOVA tests were used to determine whether differences in the measurements between LtEc species were statistically significant. Tukey's HSD test was used to determine which species differed significantly when differences between samples were determined by one-way ANOVA. An alpha value of 0.05 was used to determine statistical significance. For statistical analysis of the difference between PDA-LtEc and other species of uncoated and coated LtEc (LtEc, PEG-LtEc and Odex LtEc) structural and biophysical properties were compared using data from the literature.

### Results and Discussion

TABLE 1
Structural and biophysical properties of LtEc, PDA-LtEc, and PEG-LtEc-40%
(40% PEG surface coverage) synthesized with different reaction times from 2-16 hours.

| | Hydrodynamic | Zeta Potential | | | Cooperativity |
|---|---|---|---|---|---|
| Species | Diameter (nm) | (mV) | K_{off}, O₂ | P₅₀ (mmHg) | (n) |
| LtEc | 28.20 ± 9.41 | −30.4 ± 2.3 | 29.19 ± 0.04 | 24.86 ± 2.84 | 2.92 ± 0.39 |
| PEG-LtEc-40% | 36.00 ± 8.93 | −12 ± 1.6* | 20.36 ± 0.22* | 19.64 ± 1.12 | 1.43 ± 0.18* |
| Odex-LtEc | 44 ± 3.16* | −18.89 ± 4.00* | 23.71 ± 1.44* | 10.83 ± 1.38* | 1.56 ± 0.18* |
| PDA-LtEc 2 h | 34.71 ± 4.59 | −18.69 ± 1.52* | 31.90 ± 4.14 | 26.04 ± 2.16 | 3.38 ± 0.25 |
| PDA-LtEc 5 h | 35.97 ± 5.42 | −7.16 ± 4.16* | 30.86 ± 0.18 | 26.51 ± 6.03 | 2.57 ± 0.04 |
| PDA-LtEc 16 h | 57.54 ± 4.29* | −19.42 ± 6.28* | 28.9 ± 3.9 | 25.39 ± 4.19 | 2.02 ± 0.17* |

*Indicates a statistically significant difference (p < 0.05) from native LtEc.

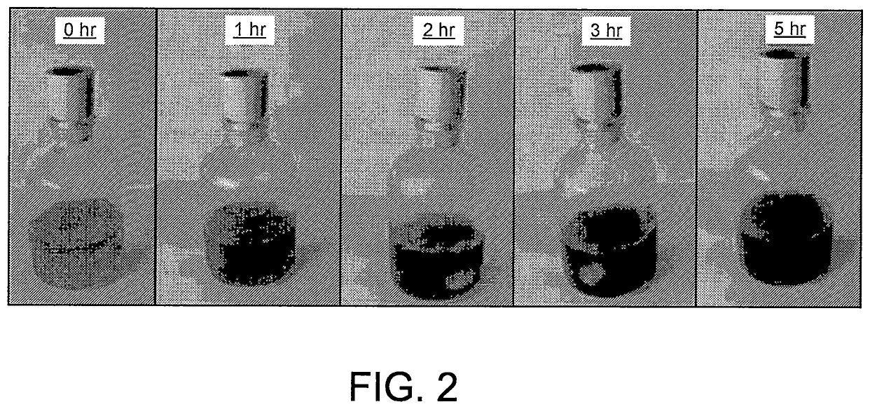

Structural Analysis. In this example, the surface of LtEc was coated with PDA to potentially mask the protein in circulation from immune detection with the aim of increasing the in vivo circulatory half-life of the HBOC. LtEc has been surface conjugated with poly-ethylene glycol (PEG) to increase the circulatory half-life of LtEc. Other studies have demonstrated that surface coating Hb nanoparticles with PDA resulted in a reduction in the in vivo immune response. As was observed with PDA-coated Hb (PDA-Hb), the progress of the PDA-LtEc synthesis reaction was observable by a qualitative change in the color and opacity of the solution during the reaction (FIG. 2). As reaction progressed, the solution darkened in color from a clear red color to an opaque dark brown hue. The degree of surface coating was visibly discernable by the change in color of the solution with increasing levels of reaction time. PDA has a characteristic black/brown color that may be observed in the darkening of the PDA-LtEc solution with increasing reaction time. This change in color is consistent with the PDA-coating polymerization process observed in previous studies involving PDA-coating of human Hb. Furthermore, we also performed the conjugation of PDA to LtEc under basic pH conditions (~pH 8.5) in the absence of the photocatalyst. This synthetic route facilitates self-polymerization of DA on a protein surface under basic pH conditions. However, we observed dissociation of the LtEc superstructure into dodecamer, trimer, and monomer subunits of LtEc. LtEc dissociates into its protomeric components under pH conditions due to the breakage of hydrogen bonds that hold the superstructure together, while maintaining disulfide linkages between monomeric and linker subunits. Hence, this necessitated the use of the photocatalytic method to conjugate PDA to the surface of LtEc.

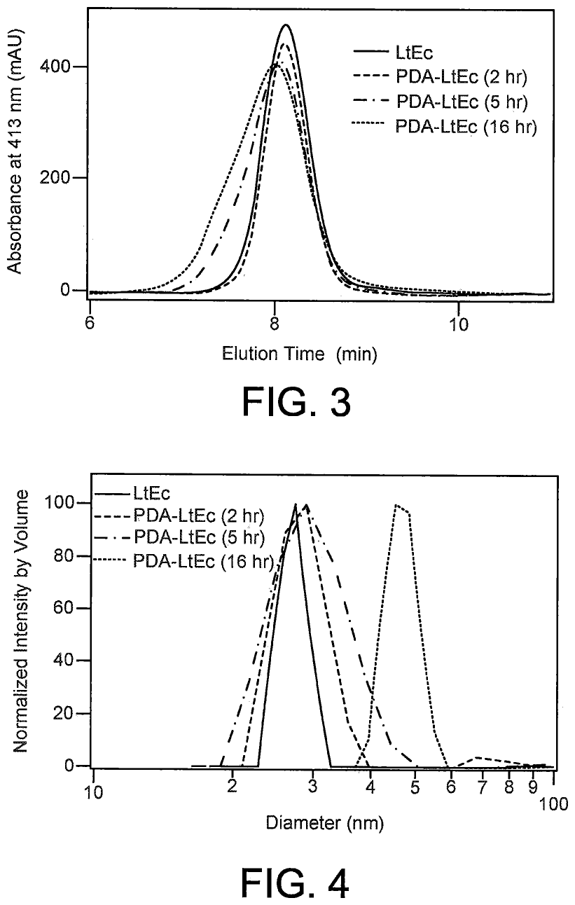

The change in the size of LtEc before and after the PDA coating process for different levels of PDA polymerization was assessed using SEC-HPLC (FIG. 3). The peak elution time of unmodified LtEc before the addition of DA was 8.2 minutes. Following the polymerization reaction to form PDA-LtEc, there was a left shift in the peak elution time and a broadening left tail of the peak, indicating a greater distribution of larger-sized particles compared to LtEc. These changes in the elution chromatogram were more pronounced with increasing reaction time. While no change in peak time or distribution was observed for PDA-LtEc that was reacted for 2 hours compared to LtEc, a slight left shift in peak elution time and broadened left tail in the peak was observed for the 5-hour reaction and more pronounced for the 16-hour reaction. A single elution peak was observed for all PDA-LtEc species (2, 5, 16 hours) indicating the presence an oligomeric structure, without the presence of dissociated monomeric units. This demonstrates that the mega-structure of LtEc remains stable during the photocatalytic polymerization of PDA and undergoes a change in particle size.

Changes in hydrodynamic diameter with PDA polymerization were further investigated by changes in measured particle diameter via DLS. (FIG. 4, Table 1). Particle diameter measurement by DLS revealed similar trends to those observed by SEC-HPLC. Native LtEc has a measured diameter of ~27 nm, while it was observed that PDA-LtEc reacted at 2 hours and 5 hours had measured diameters of 34.71±4.59 nm and 35.97±5.42 nm, respectively. These differences did not surmount to a statistically significant change following statistical analysis (p>0.05). However, it may be noted that similarly to SEC-HPLC analysis, there is a broadened right tail in particle diameter distribution and higher polydispersity index, indicating a greater prevalence of larger sized particles among PDA-LtEc (2 and 5 hours) compared to native LtEc.

PDA-LtEc reacted for 16 hours, however, had a measured particle diameter of 57.54±4.29 nm, which was significantly different than LtEc and other species of PDA-LtEc (p<0.05). These trends indicate increasing PDA-LtEc particle diameter and wider particle size distribution with increasing PDA polymerization time. Furthermore, these results also confirm the increase in thickness of the PDA coating with increasing reaction time, thus suggesting that the photocatalytic polymerization reaction works similarly to the pH-based self-polymerization reaction. Lower levels of PDA surface coating (2 and 5 hours) were not found to be statistically significantly larger than other surface-coated/conjugated LtEcs such as PEG-conjugated LtEc and oxidized dextran-conjugated-LtEc (Odex-LtEc) synthesized by our group (p>0.05). However, PDA-LtEc reacted for 16 hours was found to be significantly larger than PEG-LtEc (36.00±8.93 nm, p<0.05) but not Odex-LtEc (44±3.16 nm, p>0.05). This data demonstrates the potential to control the polymerization reaction conditions to tune the size of PDA-LtEc for optimal characteristics.

The ability to tune the size of PDA-LtEc is desirable for a potential HBOC due to the importance of the relationship between particle size and circulation time and clearance. HBOCs must be sufficiently large to prevent extravasation into the tissue space and subsequent vasoconstriction caused by unmodified hHb. As PDA-LtEc maintains the large oligomeric structure of LtEc, the surface-coated protein is sufficiently large to prevent tissue extravasation. Nanoparticle size has also been shown to affect the immune response and circulatory lifetime, with nanoparticles below −70 nm tending to accumulate in the liver while those larger than 200 nm are filtered by the spleen. PEG conjugation to the surface of LtEc results in an increase in particle size and molecular weight (MW) with an increase in oxygen affinity (i.e., reduction in P₅₀) and reduction in oxygen-binding cooperativity. When tested in golden Syrian hamsters, PEG-LtEc did not show toxic side-effects, maintained blood flow and heart rate, and showed increased circulatory life compared to unmodified LtEc. However, with increasing concern surrounding anti-PEG antibodies and PEGylated drugs, the need for a biocompatible polymer such as PDA that can also improve circulatory half-life is paramount. The controlled polymerization reaction of PDA on the surface of LtEc may allow for the engineering of a PDA-LtEc species of desirable size and biophysical properties for optimal circulation. Furthermore, the PDA-coating can be used as a building-block to assemble multi-layered nanoparticles. PDA-coatings have been also used in the layer-based assembly of PEGylated Hb nanoparticles (Hb/PEG-NPs).

The final structural analysis conducted on PDA-LtEc samples was measurement of surface charge using a zeta potential analyzer. The surface potentials of PDA-LtEc species are listed in Table 1. For PDA-LtEc species reacted for 2, 5, and 16 hours, the measured zeta potentials were −18.69±1.52 mV, −17.16±4.16 mV, and −19.42±6.28 mV respectively. All PDA-LtEc species varied significantly from the zeta potential of native LtEc (−30.4±2.3 mV, p<0.05), but were not statistically significant from other surface-modified LtEc (PEG-LtEc=−12±1.6 mV, Odex-LtEc=−18.89±4.00 mV, p>0.05).

The zeta potential represents the electrical charge at the surface of particles suspended within a fluid. The particle surface charge influences the interaction of nanoparticles in the circulation and contribute to the uptake and fate of these particles. The negative zeta potential of RBCs (~−15 mV,) affects the behavior of these cells in circulation and contributes to the repulsive forces that prevent aggregation of these cells. Native LtEc has a comparatively greater negative charge of −30.4±2.3 mV compared to RBCs. Previous literature has demonstrated that increasing the absolute magnitude of the zeta potential (positive or negative) is associated with increased phagocytic uptake and removal from the circulation.

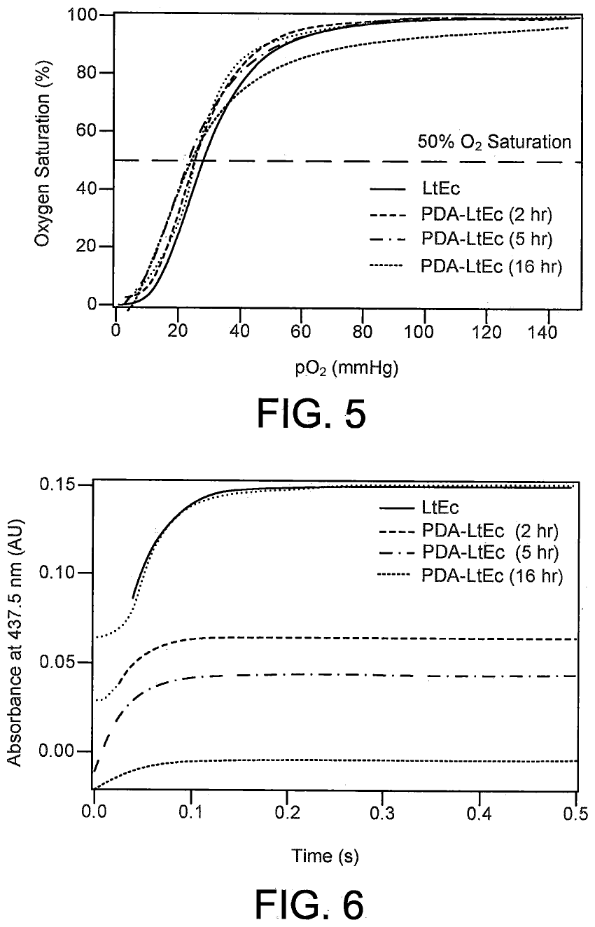

Biophysical Characterization. LtEc (16 hours) was significantly reduced compared to LtEc (p<0.05). Oxygen equilibrium analysis of PDA-LtEc demonstrated that that increasing levels of PDA polymerization did not result in a statistically significant change in the measured P₅₀ (p>0.05). The P₅₀ for PDA-LtEc (2, 5, 16 hour) was 26.04±2.16, 26.51±6.03, and 25.39±4.19 mmHg respectively, none of which varied greatly from the P₅₀ (28.68 mmHg±2.84) of native LtEc (p=0.94) (FIG. 5). While no changes in the P₅₀ were observed, oxygen equilibrium analysis showed that increasing levels of reaction time for PDA polymerization resulted in statistically significant reductions in the O₂-binding cooperativity of the molecule (p=0.003). While the cooperativity coefficient of PDA-LtEc (2 hours) was not greatly reduced compared to native LtEc (n=3.38±0.25 versus 2.92±0.39, p>0.05), the cooperativity of PDA-LtEc at both 5- and 16-hour reaction times varied significantly from the 2-hour reaction time (2.57±0.04 and 2.02±0.17, p<0.05).

The oxygen-binding properties of PDA-LtEc differ from previously synthesized surface-conjugated proteins such as PEG-LtEc and Odex-LtEc. Biophysical characterization of PEG-LtEc and Odex-LtEc revealed significantly reduced P₅₀s (19.64±1.12 and 10.83±1.38 mmHg, p<0.05) and cooperativity coefficients (1.43±0.18 and 1.56±0.18, p<0.05) respectively compared to native LtEc. The significantly different biophysical properties of PDA-LtEc compared to PEG-LtEc and Odex-LtEc may lie in the differences in the size of the monomer units that comprise the PDA, PEG, and Odex surface coatings. The mPEG-maleimide used for the surface conjugation of LtEc consisted of long mono-functionalized 5 kDa polymer chains. It has been shown that PEG chains create a large hydration layer on the surface of proteins and nanoparticles, thus hindering conformational changes in the globins in Hb upon oxygen binding. A reduction in oxygen-binding cooperativity and increase in oxygen affinity (i.e., reduction in P₅₀) has been observed for polymerized and surface-modified Hb products which is contrary to the effects of the PDA surface coating on LtEc.

In addition to equilibrium O₂ binding measurements, the O₂ offloading rate of PDA-LtEc species were analyzed using stopped flow spectroscopy and kinetic rate constants (k_{off,O2}) were calculated by fitting the change in absorbance to an exponential decay function (FIG. 6, Table 1). PDA-LtEc reacted for 2 hours, 5 hours, and 16 hours had k_{off,O2} values of 31.90±4.14, 30.86±0.18, and 28.9±3.9 s⁻¹ respectively. None of those values were statistically significant from the 29.19±0.04 s⁻¹ k_{off,O2} of LtEc (p>0.05), which demonstrates that the rate of oxygen offloading of LtEc is not significantly impacted by the PDA surface coating. This finding differs from characterization of other surface coated LtEc species. PEG-LtEc and Odex-LtEc exhibited slower offloading of oxygen (20.36±0.22 and 23.71±1.44 s⁻¹ respectively, p<0.05) than PDA-LtEc, which can be attributed to the diffusive membrane-like behavior formed by hydration of the long polymer chains of PEG and Odex. This change in oxygen offloading behavior is not observed for PDA-LtEc. The lack of significant differences in P₅₀ and k_{off,O2} for PDA-LtEc compared with native LtEc demonstrate its potential as an oxygen therapeutic.

TABLE 2
FRAP values and ABTS^{•+} free radical scavenging ratios of LtEc and
PDA-LtEc with different levels of reaction time from 2-16 hours.
Antioxidant Capacity of PDA-LtEc

| Species | FRAP Δ Abs (mAU) | Scavenging Ratio (ABTS) |
|---|---|---|
| LtEc | 0.175 ± 0.010 | 18.3 ± 2.7 |
| PDA-LtEc 2 hours | 0.221 ± 0.057 | 26.0 ± 6.7 |
| PDA-LtEc 5 hours | 0.343 ± 0.033ª | 30.8 ± 2.6 |
| PDA-LtEc 16 hours | 0.625 ± 0.019^{a,b} | 58.2 ± 5.7ª |

ªp < 0.05 compared to LtEc, ^{b}p < 0.05 compared to PDA-LtEc (5 hours).

Antioxidant and Oxidation Reduction Assays. Hb proteins bind oxygen through the coordinated Fe²⁺ atom in the heme ligand. As Hb circulates in vivo, the functional ferrous Hb may be converted into non-functional metHb with ferric Fe³⁺ heme by either auto-oxidation or by oxidizing agents. The mechanism of Hb auto-oxidation, which may be summarized as 4(HbFe²⁺O₂)+2H⁺→4HbFe³⁺+2OH⁻+3O₂, is known to generate reactive oxygen species (ROS) O2^{·-} and OH^{·} and the strong oxidant H₂O₂ as intermediates. These reactive species may cause oxidative damage to endothelial cells directly, or through the generation of the reactive ferryl Hb from the reaction of metHb and O₂·−.³⁵ From the viewpoint of both preserving the functional ferrous Hb in circulation, and mitigating ROS generated by the auto-oxidation of Hb, HBOCs with inherent antioxidant properties are desirable. PDA is a known antioxidant, and previous studies have demonstrated the antioxidant capabilities of PDA-coated Hb species.

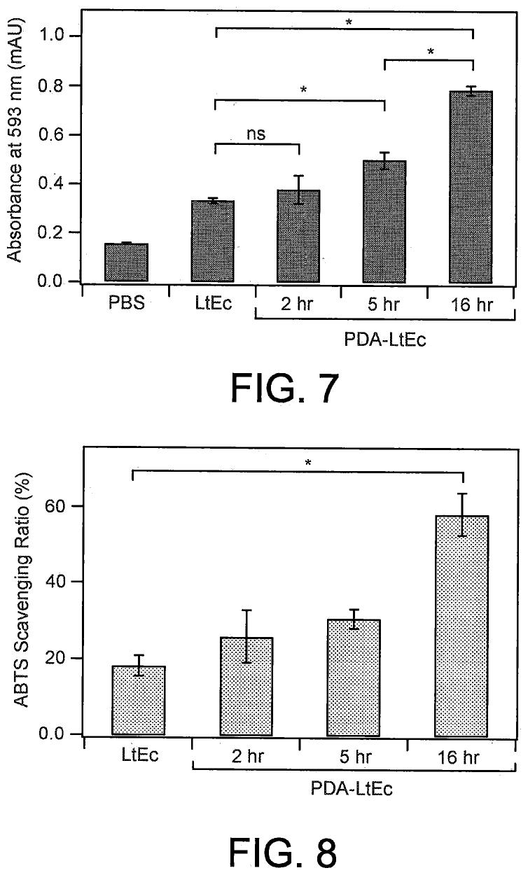

Antioxidant assays were used to evaluate the effect of the PDA-coating on the antioxidant capacity of PDA-LtEc. The FRAP assay evaluates the ability of molecules to reduce oxidized ferric ions (Fe³⁺) to ferrous ions (Fe²⁺). An increase in the measured absorbance of the FRAP solution at 593 nm after incubation with protein is caused by an increased concentration of Fe²⁺ ions, demonstrating the protein's ability to reduce ferric iron in aqueous solution. Changes in the FRAP absorbance compared to the PBS control are shown in Table 2, the measured absorbances for each sample group are shown in FIG. 7. Higher levels of polymerization in PDA-LtEc were found to be associated with statistically significant increases in absorbance compared with the 0.175±0.010 mAU increase for LtEc: an increase in absorbance of 0.343±0.033 mAU was observed for PDA-LtEc (5 hours) and 0.625±0.019 mAU for PDA-LtEc (16 hours). These levels of PDA surface coating were also statistically significant from each other, indicating increasing antioxidant capacity with increasing levels of PDA polymerization and further demonstrating the tunability of PDA-LtEc with changing the extent of PDA polymerization. This demonstrated that the ferric iron reduction capability may be useful in reducing the auto-oxidation rate of Hb or in scavenging oxidants that might either induce the formation of metHb or be generated as a by-product of its formation such as H₂O₂ or ferryl Hb.

The ABTS assay evaluates the free radical scavenging potential of antioxidants by monitoring the reduction of ABTS^{·+} cations in solution after incubation with the protein of interest in the dark. This change in free radicals is measured by the absorbance reduction of the ABTS stock solution at 734 nm and normalized to a scavenging ratio calculated by Equation 2. This assay demonstrated a statistically significant difference in the scavenging ratio of PDA-LtEc at different reaction times and LtEc (p<0.001). Scavenging ratio (SR) calculations from the ABTS assay are listed in Table 2 and FIG. 8. Tukey's HSD revealed a statistically significant difference in the scavenging ratio of the longest-reacted PDA-LtEc (t=16 hours, SR=58.2±5.7) compared to native LtEc (SR=18.3±2.7) and PDA-LtEc synthesized at shorter reaction times (t=2, 5 hours; SR=26.0±6.7, 30.8±2.6). This assay demonstrates that increasing the extent of PDA coating on the surface of LtEc acts as an antioxidant that has increasing free radical scavenging activity and further corroborates the indication of the FRAP assay that PDA-coating of LtEc provides some antioxidant capability.

Other studies have demonstrated broader implications for the antioxidant properties of HBOCs. PDA-coated Hb microparticles (PD-HbMPs) were shown to provide increased protection against oxidation, with significantly reduced generation of metHb after the addition of H₂O₂. In another instance, co-incubation of PDA-coated hHb (Hb-PDA) was found to significantly reduce the generation of ROS inside cells after the addition of H₂O₂. The antioxidant properties of PDA-LtEc demonstrates the potential for similar oxidative protection, both for the circulating protein and surrounding tissues. Thus, antioxidant capacity is property that may be desirable for engineered HBOCs.

### CONCLUSION

PDA surface coated LtEc was larger in size compared to native LtEc. This supports the efficacy of the photoredox catalytic approach for polymerization of DA into PDA for surface coating LtEc and other pH and oxidation sensitive biomolecules. Furthermore, we demonstrated that self-polymerization of DA allows control over the thickness of the PDA surface coating. This opens future work towards optimization of PDA coating thickness to optimize parameters such as particle size, surface charge, and other biophysical parameters such as oxygen-binding equilibria and offloading kinetics.

The measured differences between LtEc and PDA-LtEc highlight properties that may make PDA-LtEc an intriguing candidate for future research as a potential HBOC candidate. The results of the antioxidant activity assays in this study demonstrate the antioxidant capacity of PDA-LtEc, which may be useful for alleviating the oxidative stress common among unencapsulated HBOCs or in reducing the rate of metHb formation due to the presence of chemical oxidants in the circulation.

### REFERENCES

1. Plasma, Platelets and Whole Blood|Red Cross Blood Services. https://www.redcrossblood.org/donate-blood/how-to-donate/types-of-blood-donations/blood-components.html.
2. Mitra, R., Mishra, N. & Rath, G. P. Blood groups systems. Indian J Anaesth 58, 524 (2014).
3. Chapman, J. F. et al. Guidelines for compatibility procedures in blood transfusion laboratories. Transfusion Medicine 14, 59-73 (2004).
4. Faithfull, N. S. Artificial oxygen carrying blood substitutes. Adv Exp Med Biol 317, 55-72 (1992).
5. Buehler, P. W. & Alayash, A. I. Toxicities of hemoglobin solutions: in search of in-vitro and in-vivo model systems. Transfusion (Paris) 44, 1516-1530 (2004).
6. Silverman, T. A., Weiskopf, R. B. & Speakers, for the P. C. and the. Hemoglobin-based Oxygen Carriers: Current Status and Future Directions. Anesthesiology 111, 946-963 (2009).
7. Natanson, C., Kern, S. J., Lurie, P., Banks, S. M. & Wolfe, S. M. Cell-Free Hemoglobin-Based Blood Substitutes and Risk of Myocardial Infarction and Death: A Meta-analysis. JAMA 299, 2304-2312 (2008).
8. Schaer, D. J., Buehler, P. W., Alayash, A. I., Belcher, J. D. & Vercellotti, G. M. Hemolysis and free hemoglobin revisited: exploring hemoglobin and hemin scavengers as a novel class of therapeutic proteins. Blood 121, 1276-84 (2013).
9. Belcher, D. A., Cuddington, C. T., Martindale, E. L., Pires, I. S. & Palmer, A. F. Controlled Polymerization and Ultrafiltration Increase the Consistency of Polymerized Hemoglobin for Use as an Oxygen Carrier. Bioconjug Chem 31, 605-621 (2019).
10. Matsuhira, T. & Sakai, H. Entropy-Driven Supramolecular Ring-Opening Polymerization of a Cyclic Hemoglobin Monomer for Constructing a Hemoglobin-PEG Alternating Polymer with Structural Regularity. Biomacromolecules 22, 1944-1954 (2021).
11. Kure, T. & Sakai, H. Preparation of Artificial Red Blood Cells (Hemoglobin Vesicles) Using the Rotation-Revolution Mixer for High Encapsulation Efficiency. ACS Biomater Sci Eng 7, 2835-2844 (2021).
12. Hickey, R. & Palmer, A. F. Synthesis of Hemoglobin-Based Oxygen Carrier Nanoparticles By Desolvation Precipitation. Langmuir 36, 14166-14172 (2020).
13. Savla, C. et al. Purification of Lumbricus terrestris Mega-Hemoglobin for Diverse Oxygen Therapeutic Applications. ACS Biomater Sci Eng 6, 4957-4968 (2020).
14. Jani, V. P. et al. Polyethylene Glycol Camouflaged Earthworm Hemoglobin. Polyethylene Glycol Camouflaged Earthworm Hemoglobin. PLoS ONE 12, 170041 (2017).
15. Savla, C. & Palmer, A. F. Structural Stability and Biophysical Properties of the Mega-Protein Erythrocruorin Are Regulated by Polyethylene Glycol Surface Coverage. Biomacromolecules 22, 2081-2093 (2021).
16. Zhang, C. et al. CuSO₄/H₂O₂-Induced Rapid Deposition of Polydopamine Coatings with High Uniformity and Enhanced Stability. Angew Chem Int Ed Engl 55, 3054-3057 (2016).
17. Jia, L. et al. Polydopamine-assisted surface modification for orthopaedic implants. J Orthop Translat 17, 82-95 (2019).
18. Bispo, J. A. C., Landini, G. F., Santos, J. L. R., Norberto, D. R. & Bonafe, C. F. S. Tendency for oxidation of annelid hemoglobin at alkaline pH and dissociated states probed by redox titration. Comparative Biochemistry and Physiology—B Biochemistry and Molecular Biology 141, 498-504 (2005).
19. Wang, J., Ma, G., Huang, W. & He, Y. Visible-light initiated polymerization of dopamine in a neutral environment for surface coating and visual protein detection. Polym Chem 9, 5242-5247 (2018).
20. Elmer, J. et al. Hypervolemic infusion of Lumbricus terrestris erythrocruorin purified by tangential-flow filtration. Transfusion (Paris) 52, 1729-1740 (2012).
21. Wang, Q. et al. Bioinspired Polydopamine-Coated Hemoglobin as Potential Oxygen Carrier with Antioxidant Properties. Biomacromolecules 18, 1333-1341 (2017).
22. Benzie, I. F. F. & Devaki, M. The ferric reducing/antioxidant power (FRAP) assay for non-enzymatic antioxidant capacity: Concepts, procedures, limitations and applications. in Measurement of Antioxidant Activity and Capacity: Recent Trends and Applications 77-106 (Wiley, 2017). doi:10.1002/9781119135388.ch5.
23. Hayes, W. A., Mills, D. S., Neville, R. F., Kiddie, J. & Collins, L. M. Determination of the molar extinction coefficient for the ferric reducing/antioxidant power assay. Anal Biochem 416, 202-205 (2011).
24. Re, R. et al. Antioxidant activity applying an improved ABTS radical cation decolorization assay. Free Radic Biol Med 26, 1231-1237 (1999).
25. Savla, C. & Palmer, A. F. Structural Stability and Biophysical Properties of the Mega-Protein Erythrocruorin Are Regulated by Polyethylene Glycol Surface Coverage. Biomacromolecules 22, 2081-2093 (2021).
26. Journal, A. I. et al. Artificial Cells, Nanomedicine, and Biotechnology Influence of polydopamine-mediated surface modification on oxygen-release capacity of haemoglobin-based oxygen carriers Influence of polydopamine-mediated surface modification on oxygen-release capacity of haemoglobin-based oxygen carriers. (2018) doi:10.1080/21691401.2018.1459636.
27. Xu, F. et al. Long-circulation of hemoglobin-loaded polymeric nanoparticles as oxygen carriers with modulated surface charges. Int J Pharm 377, 199-206 (2009).
28. Lubich, C. et al. The Mystery of Antibodies Against Polyethylene Glycol (PEG)—What do we Know? Pharm Res 33, 2239-2249 (2016).
29. Liu, X. et al. Low-Fouling Electrosprayed Hemoglobin Nanoparticles with Antioxidant Protection as Promising Oxygen Carriers. Macromol Biosci 20, 1900293 (2020).
30. He, C., Hu, Y., Yin, L., Tang, C. & Yin, C. Effects of particle size and surface charge on cellular uptake and biodistribution of polymeric nanoparticles. Biomaterials 31, 3657-3666 (2010).
31. Silva, D. C. N. et al. Optical Tweezers as a New Biomedical Tool to Measure Zeta Potential of Stored Red Blood Cells. PLoS One 7, 31778 (2012).
32. Tokumasu, F., Ostera, G. R., Amaratunga, C. & Fairhurst, R. M. Modifications in erythrocyte membrane zeta potential by Plasmodium falciparum infection. Exp Parasitol 131, 245-251 (2012).
33. Fernandes, H. P., Cesar, C. L. & Barjas-Castro, M. de L. Electrical properties of the red blood cell membrane and immunohematological investigation. Rev Bras Hematol Hemoter 33, 297-301 (2011).
34. Faivre, B., Menu, P., Labrude, P. & Vigneron, C. Hemoglobin Autooxidation/Oxidation Mechanisms and Methemoglobin Prevention or Reduction Processes in the Bloodstream Literature review and outline of autooxidation reaction. http://dx.doi.org/10.3109/10731199809118943. 26, 17-26 (2009).
35. Hai, C.-M. Systems Biology of HBOC-Induced Vasoconstriction. Curr Drug Discov Technol 9, 204 (2012).
36. Baidukova, O. et al. Antioxidative protection of haemoglobin microparticles (HbMPs) by Polydopamine. Artif Cells Nanomed Biotechnol 46, S693-S701 (2018).
37. Wang, Q. et al. Influence of polydopamine-mediated surface modification on oxygen-release capacity of haemoglobin-based oxygen carriers https://doi.org/10.1080/21691401.2018.1459636. 46, 484-492 (2018).

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A method for encapsulating a pH sensitive cargo in a polydopamine shell, the method comprising:
contacting the cargo with dopamine monomers and a photoredox catalyst to form a reaction mixture; and
irradiating the reaction mixture with actinic radiation, thereby polymerizing the dopamine monomers to form a shell of polydopamine encapsulating the cargo.

2. The method of claim 1, wherein the cargo comprises a nanoparticle or microparticle.

3. The method of claim 1, wherein the cargo comprises a biomolecule.

4. The method of claim 3, wherein the biomolecule comprises a protein.

5. The method of claim 4, wherein the protein comprises an oxygen binding protein.

6. The method of claim 5, wherein the oxygen binding protein is chosen from hemoglobin, myoglobin, erythrocruorin, hemocyanin, hemerythrin, chlorocruorin, vanabin, pinnaglobin, leghemoglobin, a polymerized version thereof and/or a polymer-conjugated version thereof.

7. The method of claim 1, wherein the cargo comprises an erythrocruorin, such as an annelid erythrocruorin.

8. The method of claim 1, wherein contacting the cargo with the dopamine monomers and the photoredox catalyst comprises stirring a solution or dispersion comprising the cargo, the dopamine monomers, and the photoredox catalyst.

9. The method of claim 8, wherein the solution or dispersion comprises an aqueous solution or dispersion.

10. The method of claim 9, wherein the aqueous solution or dispersion has a pH of from 6 to 8, such as a pH of from 6.5 to 7.4.

11. The method of claim 1, wherein the photoredox catalyst is chosen from N-(4-(10H-phenothiazin-10-yl)phenyl)acrylamide, perylene, perylene diimide, 10-phenylphenothiazine, 2,3-dichloro-5,6-dicyano-p-benzoquinone, eosin Y, fluorescein, rose Bengal, methylene blue, 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, bis(2,2′-bipyridine)-(5-aminophenanthroline)ruthenium bis(hexafluorophosphate), tris(bipyridine)ruthenium(II) chloride, tris(bipyridine)ruthenium(II) hexafluorophosphate, tris-(bipyrazine)ruthenium(II) hexafluorophosphate, tris-(phenanthroline)ruthenium(II) chloride, tris-(bipyrimidine)ruthenium(II) chloride, bis-(2-(2′,4′-difluorophenyl)-5-trifluoromethylpyridine)(di-tert-butylbipyridine)iridium(III) hexafluorophosphate, bis-(2-phenylpyridine)(di-tert-butylbipyridine)iridium(III) hexafluorophosphate, and bis(2,9-di(para-anisyl)-1,10-phenanthroline)copper(I) chloride, or a combination thereof.

12. The method of claim 11, wherein the photoredox catalyst is 9-mesityl-2,7-dimethyl-10-phenylacridinium tetrafluoroborate, 9-mesityl-10-methylacridinium perchlorate, 9-mesityl-10-methylacridinium tetrafluoroborate, 9-Mesityl-10-phenylacridinium tetrafluoroborate, or a combination thereof.

13. The method of claim 1, wherein the actinic radiation has a wavelength of from 200 nm to 1000 nm.

14. The method of claim 1, wherein the reaction mixture is at a temperature of from 20° C. to 30° C.

15. The method of claim 1, wherein the method further comprises, following irradiation, filtering the cargo encapsulated in the polydopamine shell by ultrafiltration against a filtration membrane having a pore size that separates low molecular weight contaminants from the cargo encapsulated in the polydopamine shell.

16. The method of claim 15, wherein the ultrafiltration comprises tangential-flow filtration.

17. The method of claim 15, wherein the filtration membrane is rated for retaining solutes having a molecular weight greater than 100 kDa, thereby forming a retentate fraction comprising the cargo encapsulated in the polydopamine shell and a permeate fraction comprising low molecular weight contaminants having a molecular weight less than 100 kDa.

18. A method for making a polydopamine-encapsulated oxygen carrier, the method comprising:
contacting an oxygen binding protein with dopamine monomers and a photoredox catalyst to form a reaction mixture; and
irradiating the reaction mixture with actinic radiation, thereby polymerizing the dopamine monomers to form the polydopamine-encapsulated oxygen carrier.

19-31. (canceled)

32. A polydopamine-encapsulated oxygen carrier made by the method of any of claim 1.

33. (canceled)

34. A particle comprising an erythrocruorin encapsulated in a polydopamine shell.

35-37. (canceled)

38. A method of treating a subject comprising administering to the subject the polydopamine-encapsulated oxygen carrier defined by claim 32.

39. (canceled)
